(19) United States
(12) Patent Application Publication
(10) Pub. No.: US 2024/0091453 A1
(43) Pub. Date: Mar. 21, 2024

(54) Electronic System for a Drug Delivery Device and Drug Delivery Device

(71) Applicant: Sanofi, PARIS (FR)

(72) Inventors: Oliver Charles Gazeley, Basel (CH); Adam Moyo Harvey-Cook, Billericay, Essex (GB); Craig Ashley Mason, Helsby (GB); Aidan Michael O`Hare, Warwick (GB); David Aubrey Plumptre, Droitwich Spa (GB); Maurice Toporek, Frankfurt am Main (DE); Robert Veasey, Warwick (GB)

(21) Appl. No.: 18/273,707

(22) PCT Filed: Jan. 26, 2022
(86) PCT No.: PCT/EP2022/051672

§ 371 Date: Jul. 21, 2023

(30) Foreign Application Priority Data
Jan. 28, 2021 (EP) 21315011.3

(51) Int. Cl. A61M 5/315

(52) U.S. Cl. CPC A61M 5/31548; A61M 5/31566; A61M 5/31585; A61M 2205/13; A61M 2205/502; A61M 2209/04

(57) ABSTRACT

An electronic system for a drug delivery device is provided, the electronic system comprising at least one user interface member configured to be manipulated by a user for performing a dose operation, e.g. a dose setting operation to set a dose of drug to be delivered by the drug delivery device and/or a dose delivery operation for delivering a set dose, an electronic control unit, the electronic control unit being configured to control operation of the electronic system, the electronic system having a first state and a second state, wherein the electronic system has an increased electrical power consumption in the second state as compared to the first state, wherein the user interface member comprises an exterior operation surface which is arranged to be touched by the user for the dose operation, wherein the user interface member comprises a user proximity detection unit, wherein the user proximity detection unit is configured to generate an electrical signal when the user is close to the exterior operation surface or touches the exterior operation surface, wherein the user proximity detection unit comprises a movable member, wherein the movable member is arranged to be moved by the user away from an initial position relative to the exterior operation surface towards an operation position before the user reaches the exterior operation surface, wherein the user proximity detection unit further comprises an electrical signaling unit, wherein the user proximity detection unit is configured to provide the electrical signal when the movable member has been moved away from the initial position, e.g. when the movable member is in the operation position or during the movement away from the initial position towards the operation position, and wherein the electronic system is configured such that the electronic control unit switches the electronic system from the first state into the second state in response to the electrical signal.

### CROSS REFERENCE TO RELATED APPLICATIONS

The present application is the national stage entry of International Patent Application No. PCT/EP2022/051672, filed on Jan. 26, 2022, and claims priority to Application No. EP 21315011.3, filed on Jan. 28, 2021, the disclosures of which are incorporated herein by reference.

### TECHNICAL FIELD

The present disclosure relates to an electronic system for a drug delivery device. The present disclosure further relates to a drug delivery device, which preferably comprises the electronic system.

### BACKGROUND

Drug delivery devices using electronics are becoming increasingly popular in the pharmaceutical industry as well as with users or patients. However, especially if the device is designed to be self-contained, that is to say without a connector for a connection to an external electrical power source which is necessary to provide electrical power for the operation of the device, the management of the resources of a power supply integrated into the device is particularly important, while maintaining a reliable operability of the system.

### SUMMARY

Certain aspects of the present disclosure relate to a novel, expediently improved electronic system or drug delivery device.

One aspect of the present disclosure relates to an electronic system for a drug delivery device. Another aspect of the present disclosure relates to a drug delivery device, preferably one comprising the electronic system. Accordingly, the features, which are disclosed in relation to the drug delivery device or units thereof or therefore, do also apply for the electronic system and vice versa.

In one embodiment, the electronic system comprises at least one user interface member. The user interface member may be arranged or configured to be manipulated, e.g. touched and/or moved, by a user, e.g. the user of the drug delivery device such as the patient. The user interface member may be provided for performing a dose operation, e.g. a dose setting operation to set a dose of drug to be delivered by the drug delivery device and/or a dose delivery operation for delivering a set dose, preferably the dose which has been previously set during the dose setting operation. The dose operation may be an operation which is related to the dose which is to be delivered by the drug delivery device, either to the dose delivery operation or to the dose setting operation. The dose operation, in addition to the dose setting operation, may comprise a dose adjusting operation, where a previously set dose, which expediently has not been delivered yet, is either increased or decreased by manipulating the user interface member. Performing the dose operation may require continuous contact of the user to the user interface member, particularly to an exterior operation surface thereof (see below), throughout the entire operation. The dose operation may involve movement of the user interface member relative to a housing of the drug delivery device, e.g. a housing of a drug delivery device unit to which the electronic system can be connected or a housing of the drug delivery device.

In one embodiment, the user interface member comprises an exterior operation surface. The exterior operation surface may be an exterior surface of the user interface member. The exterior operation surface may be arranged and/or configured to be touched by the user, particularly for the dose operation. It may be necessary to touch the surface in order to perform the dose operation, e.g. to initiate the operation, and/or to maintain contact with the exterior operation surface during the entire dose operation to complete the dose operation. The exterior operation surface may be a setting surface for the dose setting operation and/or a delivery surface for the dose delivery operation. The setting surface and the delivery surface may face in different directions. The setting surface may face in the radial direction. The delivery surface may face in an axial direction, e.g. in a proximal direction. The setting surface may extend circumferentially around an axis of the user interface member, e.g. a rotation axis around which the user interface member is rotatable for a dose setting operation. The delivery surface may be arranged obliquely or perpendicularly relative to the axis. The setting surface may also be used for the dose adjusting operation.

In one embodiment, the electronic system comprises an electronic control unit. The electronic control unit may be configured to control an operation of the electronic system. The electronic control unit may be or may comprise an electronic processor, such as a microcontroller or an ASIC, for example. The electronic system may have a first state, e.g. when the system is dormant or idle, and a second state, e.g. when the system is operational. The electronic system may have an increased electrical power consumption in the second state as compared to the first state. In the first state, one or more electrical or electronic units of the electronic system may be in a sleep mode or be powered off such that they have no significant power consumption or no power consumption. For example, in the second state, a motion sensing unit may be active, i.e. it can be operated or does operate, where this unit is not active, i.e. it cannot be operated or does not operate, in the first state. The motion sensing unit will be described in more detail below. Alternatively or additionally, a communication unit may be inactive in the first state and active in the second state. The communication unit will be described in more detail below.

In one embodiment, the electronic system, preferably the user interface member, comprises a user proximity detection unit. The user proximity detection unit may be operatively connected to the electronic control unit. The user proximity detection unit may be configured to generate an electrical signal, e.g. a use signal, when the user is close to the exterior operation surface of the user interface member or touches the exterior operation surface. The electrical signal may be indicative or may be regarded as being indicative, e.g. by the electronic control unit, that the dose operation is about to be commenced.

In one embodiment, the electronic system comprises a movable member. The movable member may be part of the user interface member. The movable member may be part of the user proximity detection unit. The exterior operation surface of the user interface member may be formed by a user interface member body. The movable member may protrude from and/or a contact region of the movable member may be elevated relative to the exterior operation surface of the user interface member, e.g. at least in the initial position. The movable member may be configured and arranged relative to the exterior operation surface such that the movable member has to be moved before the user can touch the exterior operation surface, e.g. with the thumb.

In one embodiment, before the user reaches the exterior operation surface, the movable member has to be moved away from an initial position towards and preferably into an operation position relative to the exterior operation surface.

In one embodiment, the electronic system comprises an electrical signaling unit. The signaling unit may be operatively connected to the electronic control unit. The signaling unit may be part of the user proximity detection unit. The movable member may be designed to cooperate with the signaling unit, e.g. mechanically, to trigger generation of the electrical signal. The signaling unit may be configured to provide an electrical signal, such as the electrical signal which the user proximity detection unit is configured to generate, wherein the signal is indicative for the user being close to or touching the exterior operation surface.

In one embodiment, the signaling unit is secured relative to the exterior operation surface, e.g. against movement away from and/or towards the exterior operation surface. The signaling unit may be rotationally and axially fixed relative to the exterior operation surface.

In one embodiment, the user proximity detection unit is configured to provide the signal when, preferably only when, the movable member has been moved away from the initial position, e.g. towards the operation position. The user proximity detection unit may be configured to provide the signal in response to movement of the movable member away from the initial position. The user proximity detection unit may be configured to provide the signal when, preferably only when, the movable member is in the operation position or during the movement away from the initial position towards the operation position. The movable member and the signaling unit may be arranged such that the movable member cooperates with the signaling unit to trigger the signaling unit to provide the signal in response to the movement away from the initial position. The movable member may be movable relative to the exterior operation surface and/or relative to the signaling unit. Unless the movable member has been moved relative to the operation surface, towards or into the operation position no signal may be generated by the signaling unit.

In one embodiment, the movable member protrudes less from the exterior operation surface in the operation position than in the initial position. That is to say, the direction from the initial position to the operation position may be towards the exterior operation surface as seen from that end of the movable member which is remote from the exterior operation surface and arranged on the exterior of the movable member. The movement direction may be a distal direction, for example, especially if the exterior operation surface is a delivery surface.

In one embodiment, the electronic control unit is configured to switch the electronic system from the first state into the second state, e.g. by issuing an according command or signal. The electronic control unit may be configured to switch the electronic system from the first state into the second state in response to the at least one electrical signal, preferably in direct response to the signal generated in response to movement of the movable member away from the initial position. The signal provided by the user proximity detection unit may be processed by the electronic control unit, which, in response to the signal, may switch the electronic system from the first state of low power consumption to the second state of higher power consumption. The signal provided by the user proximity detection unit may be indicative for the dose operation, e.g. the dose delivery operation. The electronic control unit may be configured to switch the system, e.g. by issuing an according command or signal, into the second state in direct and/or immediate response to the receipt of the signal, e.g. receipt of just one signal pulse or just one change in an electrical characteristic, e.g. voltage or current. Complicated signal patterns of succeeding signals or changes in characteristic for initiating the switching to the second state may be avoided in this way. The signal may be a change in voltage or in current detected by the electronic control unit.

Providing the movable member as an object or obstacle which has to be moved by the user before the exterior operation surface can be touched by the user has the advantage that the position of the movable member relative to the exterior operation surface can be used as an indicator whether the user is close to or touches the surface. In this way, the electrical signal which triggers the switching of the electronic system from the first state into the second state can be provided very early during preparation of the dose operation, e.g. right before the dose operation is commenced and/or before the exterior operation surface is touched for performing the dose operation or when the exterior operation surface is touched for performing the dose operation. Thus, the signal indicative for an operation to be conducted can be produced before the operation is actually commenced. Commencing the dose operation may require moving the user interface member, e.g. axially in a distal direction, relative to a housing or other components of a drug delivery device or of a drug delivery device unit while touching the exterior operation surface. The signal is preferably generated by the signaling unit before the user moves the user interface member for the dose operation. On the other hand, requiring movement of the movable member reduces the risk of unintentional power drain as compared to non-contact switches for example which may operate without any movement.

In one embodiment, the electronic control unit is configured to switch the electronic system to the second state in response to unidirectional movement of the movable member from the initial position to the operation position, e.g. in response to distal movement or movement of a user contact region of the movable member towards the exterior operation surface.

In one embodiment, the user interface member comprises a user interface member body. The user interface member body may define or form the exterior operation surface. A portion of the movable member may be arranged, preferably movably retained, in the user interface member body. The portion of the movable member may be the portion which is displaced from the initial position towards the operation position before the user touches the exterior operation surface. In the interior of the user interface member (body), the signaling unit and/or the electronic control unit may be arranged, e.g. axially and rotationally fixed relative to the body. The interior of the user interface member body may comprise a sealed compartment which is sealed relative to the exterior, preferably at least when the electronic system is operative in the drug delivery device.

In one embodiment, the exterior operation surface delimits at least one opening laterally or circumferentially. In other words, the exterior operation surface may be interrupted by at least one opening. A portion of the movable member may protrude through the opening, e.g. from the interior to the exterior of the user interface member (body). The movable member may be operatively connectable to elements within the interior of the user interface member body, particularly the signaling unit which may be provided below the exterior operation surface. The movable member may be actuatable from the exterior such as by the user moving the movable member relative to the user interface member body. The portion of the movable member protruding through the opening may define a user contact region or surface which can be touched by the user to move the movable member relative to the user interface member body. The user contact surface may face in the same direction as the exterior operation surface, e.g. proximally. The initial position of the movable member and the operation position of the movable member may be positions of the user contact region relative to the user interface member body, e.g. axially offset positions. The movable member may be axially guided with respect to the user interface member body. That is to say it may be moved only axially relative to the user interface member body without rotating.

In one embodiment, the exterior operation surface delimits a plurality of separate openings. The openings may be circumferentially delimited by the user interface member body or the exterior operation surface around the entire circumference. That is to say the openings may be not connected. The openings may be distributed over the exterior operation surface. A portion of the movable member may protrude through each opening of the plurality of opening in the exterior operation surface. This enables various contact regions distributed over the exterior operation surface between the user and the movable member.

In one embodiment, the respective portion of the movable member may originate from a main body of the movable member. In case of a plurality of portions, the main body may be a common main body. The main body may be arranged in the interior of the user interface member body. From that main body, the respective portions may extend through the respective openings towards and beyond the exterior operation surface. The main body may be movably retained in the interior of the user interface member body.

In one embodiment, the protruding portion of the movable member protruding from the exterior operation surface or being proud of that surface in the initial position and the opening through which the movable member extends to connect the protruding portion with an interior portion of the movable member in the interior of the user interface member body are adjusted such that the entire protruding portion can be received in the opening, especially when the movable member is moved from the initial position into the operation position. This facilitates that the movable member may be flush or sub-flush with the exterior operation surface when in the operation position and/or that contact of the user to the exterior operation surface can be made easily when the movable member is in the operation position.

In one embodiment, the movable member is arranged such that the user moves the movable member, particularly by moving the portion protruding through the opening, from the initial position towards the operation position when the user attempts to touch the exterior operation surface for the dose operation. In the operation position of the movable member, the user may touch the exterior operation surface and, preferably, the movable member. In the initial position of the movable member, the user may be remote from the exterior operation surface, preferably when touching the movable member.

In one embodiment, the operation position is offset from the initial position in a direction towards the exterior operation surface as seen from the initial position, e.g. in plan view onto the exterior operation surface. In the operation position, the movable member may protrude less from the exterior operation surface than in the initial position or be flush or sub-flush relative to the exterior operation surface. The operation position may be axially, e.g. distally, offset relative to the initial position.

In one embodiment, during the dose operation, the movable member may be in the operation position, preferably during the entire dose operation. The user may maintain the movable member in the operation position relative to the exterior operation surface during the dose operation.

In one embodiment, in the initial position of the movable member, a user contact region of the movable member, expediently a surface of the portion protruding through the opening, such as a surface facing away from the exterior operation surface, is elevated relative to the exterior operation surface and/or relative to an enveloping surface defined by the exterior contour of the exterior operation surface or the user interface member body. Therefore, an elevated contact region may be provided by the movable member for making contact with the user before the user touches the exterior operation surface. The enveloping surface may cover the opening(s) and be formed by the exterior operation surface in the regions not provided with openings. In the region of the opening, the enveloping surface may continue the contour of the exterior operation surface. In the operation position of the movable member, the user contact region, preferably all user contact regions, of the movable member may be recessed relative to the exterior operation surface and/or relative to the enveloping surface, expediently in a direction away from the initial position. In other words, the user contact region may be sub-flush relative to the exterior operation surface in the operation position. The user contact region may be arranged below the exterior operation surface or offset from the initial position by a distance which is greater than the distance between the user contact region and the exterior operation surface in the initial position. The recessed arrangement of the user contact region may be achieved by the users finger being flexible and extending partly into the opening. If the movable member is recessed relative to the exterior operation surface in the operation position, the majority of a dose operation force which has to be exerted on the user interface member for performing the dose operation can be guided through the user interface member body and, advantageously, not via the movable member which may be provided to mechanically contact an electronic unit of the system. This reduces the risk of damaging the electronic control unit or other electronic or electrical units or components in the system by force being transferred to the unit from the user via the movable member.

In one embodiment, the electrical signal is generated in response to movement of the movable member relative to the signaling unit. The movable member may be moved relative to the signaling unit from a non-signaling position into the signaling position where, in the signaling position the signal generation is triggered. In the signaling position, the movable member may be in the operation position relative to the exterior operation surface or be offset from the operation position, e.g. towards the initial position.

In one embodiment, the system is configured such that the electrical signal is generated during the movement of the movable member from the initial position to the operation position relative to the exterior operation surface. Hence, signaling position and non-signaling position may be the same positions as the operation position and the initial position respectively. In this case, the signaling unit may be fixedly mounted relative to the exterior operation surface.

In one embodiment, the electrical signal is generated in response to movement of the exterior operation surface and the movable member relative to the signaling unit, preferably only when the movable member is in the operation position relative to the exterior operation surface. The relative movement may be movement towards the signaling unit, e.g. movement in the distal direction. In this case, the movable member has to be moved at first from the initial position into the operation position and, thereafter, the exterior operation surface and the movable member are displaced towards the signaling unit in order to trigger generation of the signal. In this case, the exterior operation surface may be movable relative to the signaling unit.

In one embodiment, the signaling unit is configured such that the movable member cooperates with the signaling unit in order to trigger generation of the electrical signal. In order to cooperate with the signaling unit the movable member may have to be moved away from the initial position, preferably into the operation position. That is to say, the movable member may have to be in the operation position in order to trigger generation of the electrical signal

In one embodiment, the signaling unit comprises an electrical switch. The switch may be arranged to be triggered by the movable member, e.g. when the movable member is moved from the non-signaling position into the signaling position. The switch, when triggered may cause the generation of the electrical signal.

In one embodiment, the movable member, when the movable member is in the operation position, is biased towards the initial position by a member biasing mechanism or biasing member. Thus, the initial position may be the standard position of the movable member relative to the exterior operation surface. The member biasing mechanism may be biased or loaded during the movement of the movable member from the initial position into the operation position, e.g. by force provided by the user.

In one embodiment, a member biasing mechanism is provided which acts against movement of the movable member away from the initial position and/or towards the operation position. The member biasing mechanism may be the same mechanism as the one in the preceding paragraph. The member biasing mechanism may ensure that the initial position is the standard position of the movable member and/or that the movable member protrudes from the exterior operation surface in the initial position. The biasing force of the biasing mechanism may have to be overcome to move the movable member into the operation position.

In one embodiment, the member biasing mechanism comprises a spring, e.g. a compression spring.

In one embodiment, the dose operation is the dose delivery operation.

In one embodiment, the exterior operation surface is the delivery surface, which is arranged to be touched by the user for performing the dose delivery operation. The delivery surface may be a proximally facing surface of the user interface member body. The dose delivery force applied to the surface may be a force required to perform the dose delivery operation, e.g. a force which is transferred from the user via the user interface member via one or more mechanism members of a dose setting and drive mechanism to a piston rod of the dose setting and drive mechanism. The force may be provided to drive the dispensing operation to dispense liquid from a reservoir.

In one embodiment, for performing the dose operation, a dose operation force, e.g. a distally directed force for the dose delivery operation, has to be exerted by the user onto the user interface member. The user interface member may be designed such that the part of the dose operation force acting on the exterior operation surface is greater than that part of the operation force acting on the movable member during the dose operation. The user may be in contact with both, the exterior operation surface and the movable member, during the dose operation. The dose operation force may be oriented in the same direction as the movement from the initial position into the operation position. Favoring the exterior operation surface over the movable member may be assisted, for example, by the contact area between the finger, e.g. the user's thumb, and the exterior operation surface being greater than the contact area between the finger and the movable member when the dose operation is performed and/or in the operation position of the movable member.

In one embodiment, when the movable member is in the operation position, the movable member can or could still be moved further away from the initial position, e.g. against the force of the member biasing mechanism. In other words, the electronic system may be free of an end stop for defining the operation position. The end stop would limit movement of the movable member further away from the initial position and away from the operation position. Due to the lack of the end stop and the still possible movability of the movable member further away from the initial position in the operation position a force transferred into the system via the movable member and not via the exterior operation surface is expediently small. This may assist or be responsible in favoring force transfer via the exterior operation surface and preventing excessive force being transferred via the movable member.

In one embodiment, in the operation position, the movable member is still movable against the bias of the member biasing mechanism, e.g. away from the initial position. Thus, in the operation position, the biasing force provided by the mechanism is the force transferred to the system via the movable member. The biasing force in the operation position may be reacted by the user. The biasing force in the operation position may be less than or equal to 1 N. The electronic system may be configured such that the biasing force is smaller than the remaining part (RP) of the dose operation force, i.e. the force which the user still has to exert onto the user interface member to perform the dose operation after the movable member has been moved into the operation position. The remaining part of the force may comprise contributions of a driving force (DF), i.e. a force required to drive the dose operation, and/or a clutch switch force (CSF), i.e. the force which has to be exerted to switch the state of a clutch and/or to switch a dose setting and drive mechanism from a setting configuration to a delivery configuration. The biasing force may be less than or equal to one of the following values: RP/5, RP/6, RP/7, RP/8, RP/9, RP/10, DF/5, DF/6, DF/7, DF/8, DF/9, DF/10, CSF/5, CSF/6, CSF/7, CSF/8, CSF/9, CSF/10.

In one embodiment, as seen in plan view or top view onto the exterior operation surface, the area covered by the exterior operation surface (and preferably not covered by the movable member) is greater than the area covered by the movable member, e.g. the user contact region thereof. For example, the ratio between the area covered by the movable member and the area of the exterior operation surface may be less than or equal to any one of the following values: 0.4, 0.3, 0.25, 0.2, 0.15, 0.1, 0.05, 0.01, 0.005, 0.004. Alternatively or additionally, the ratio may be greater than or equal to any one of the following values: 0.001, 0.002, 0.003, 0.004, 0.005, 0.007, 0.01.

In one embodiment, as seen in plan view or top view onto the exterior operation surface, the protruding portion of the movable member and the exterior operation surface do not overlap in the initial position and the operation position.

In one embodiment, the exterior operation surface is a continuous surface interrupted only by one or more openings.

In one embodiment, when the movable member is in the operation position, the force exerted by the user on the movable member is resolved by the user interface member body, especially via a force transfer path, which bypasses the signaling unit and/or the electronic control unit. In other words, when the movable member is in the operation position, there may be an abutment between the movable member and the user interface member body or a component rigidly secured to the body, e.g. axially and/or rotationally. The force exerted on movable member or operation surface, e.g. for the dose operation, expediently is not guided to the electronic unit, the signaling unit and/or a carrier for these units, e.g. a conductor carrier in this case as it is resolved or reacted by the user interface member body, for example.

In one embodiment, the user interface member or the electronic system is connected to or configured to be connected to, e.g. rotationally and/or axially locked to, a mechanism member of a dose setting and drive mechanism of the drug delivery device, e.g. a dose and/or injection button or a drive member such as a drive sleeve. The user interface member or the electronic system may be operatively connected to the mechanism member such that force, especially force acting on the exterior operation surface such as the dose operation force, can be transferred from the exterior operation surface to the mechanism member. The force may be a distally directed force and/or the dose operation may be the dose delivery operation. The electronic system or the user interface member, e.g. the interface member body or a part connected to the interface member body, e.g. rigidly or movably, may have a connection feature for the connection to the mechanism member.

In one embodiment, the electronic system or the user interface member comprises a shuttle member. The shuttle member may be movable relative to the exterior operation surface. The shuttle member may be movably retained in the user interface member body. The shuttle member may be axially movable relative to the user interface member body. Relative rotational movement between shuttle member and user interface member body may be prevented. The shuttle member may be splined to the user interface member body such that (e.g. limited) relative axial movement between shuttle member and user interface member body is permitted but relative rotational movement is prevented. The shuttle member may be retained within the user interface member body, e.g. such that the shuttle member cannot be removed from the user interface member body.

In one embodiment, the electronic system comprises a shuttle member biasing system. The shuttle member biasing system, e.g. a compression spring, may be arranged to bias the shuttle member away from the exterior operation surface and/or the movable member.

In one embodiment, the electronic system comprises a carrier, such as a conductor carrier, e.g. a circuit board. The electronic control unit and/or the signaling unit may be arranged on the carrier, preferably mounted on the carrier. The carrier may be fixed to the user interface member body, e.g. axially and/or rotationally. In this case, the operation position may be the signaling position. Alternatively, the carrier may be fixed to the shuttle member, such that the carrier always moves together with the shuttle member relative to the exterior operation surface.

In one embodiment, the shuttle member is arranged in the force transfer path from the exterior operation surface to the mechanism member, especially during the dose delivery operation. The exterior operation surface may have to be moved relative to the shuttle member before force can be transferred from the shuttle member to the mechanism member. The shuttle member biasing system may have to be biased before the mechanism member is or can be moved for the dose operation.

In one embodiment, the electronic system is configured such that the user interface member is operatively connectable to the mechanism member via a dose operation interface to drive movement of the mechanism member during the dose operation. The electronic system may be configured such that, in order to establish the dose operation interface, the exterior operation surface has to be displaced from a first position relative to the mechanism member and/or the shuttle member to a second position relative to the mechanism member and/or the shuttle member. The direction from the first position to the second position may be an axial direction, e.g. the distal direction and/or the direction of movement of the movable member from the initial position to the operation position. In the second position, there may be an abutment established in the force transfer path between the exterior operation surface and the shuttle member which slaves the shuttle member, e.g. axially, to the exterior operation surface or the user interface member body, e.g. by the interface member body or a component secured thereto axially abutting the shuttle member. The shuttle member biasing system may be configured to be biased during the movement from the first position to the second position. The shuttle member may be pre-loaded and the pre-load may have to be overcome for moving the exterior operation surface into the second position. The electronic system may be configured such that the signal is generated only if the movable member is in the operation position relative to the exterior operation surface, e.g. because only in that position the movable member may trigger the switch of the signaling unit. The signal may be generated before the exterior operation surface is moved towards the second position or only thereafter. The signal may be generated before the exterior operation surface of the user interface member body has reached the second position or when the second position has been reached. When the mechanism member is slaved to the user interface member body or the exterior operation surface, e.g. via the shuttle member, the dose operation can be performed by the user by moving the mechanism member via the exterior operation surface. Having the shuttle member with the shuttle member biasing system integrated into the electronic system ensures that the signal is generated in all tolerance conditions before the mechanism member is operated for the dose operation or before the piston rod moves. A defined pre-load may be provided by the shuttle member biasing system to ensure that the signal can be generated always before the dose setting and drive mechanism is switched to the dose delivery configuration.

In one embodiment, the mechanism member is a second member. The dose setting and drive mechanism may comprise a first member, e.g. a number sleeve or a dial sleeve. The first member and the second member may be configured to be movable relative to each other for switching the dose setting and drive mechanism from a dose setting configuration into a dose delivery configuration, such as by switching the state of a clutch coupling, e.g. from engaged to disengaged or vice versa. The clutch coupling when engaged may rotationally lock the first member to the second member. When the clutch coupling is released, the second member may be rotatable relative to the first member. The electronic system may be configured such that the signal is generated before the dose setting and drive mechanism is switched from the dose setting configuration to the dose delivery configuration, e.g. via the user interface member. This facilitates the electronic system being switched on timely enough to perform actions during the dose delivery operation. The force which the user has to exert to generate the electrical signal, e.g. to overcome the biasing force of the member biasing mechanism, is expediently smaller than the force which is required to be exerted on the user interface member or the exterior operation surface to switch the dose setting and drive mechanism from the dose setting configuration to the dose delivery configuration, e.g. the clutch switch force.

In one embodiment, the movable member is rigid. In this case, a separate member biasing system may have to be provided in order to bias the movable member towards its initial position when it is in the operation position.

In one embodiment, the movable member is elastically deformable. In this case, the elastic restoring force may be used to bias the movable member towards its initial position when it is in the operation position.

In one embodiment, the movable member is configured to establish a sealed interface for sealing the interior of the user interface member body from the exterior. For forming the sealed interface, a sealing portion of the movable member may be retained, e.g. clamped, between two parts of the user interface member, e.g. the body defining the exterior operation surface and a further part. The sealing portion may be elastically or plastically deformed to provide a sealing engagement with one or both of the parts.

In one embodiment, the user interface member body is rigid.

In one embodiment, the main body of the movable member is movably retained in the interior of the user interface member. The, e.g. rigid, main body may be provided with a sealing member to sealingly engage an interior wall of the user interface member. The main body may seal the interior of the user interface member body from the exterior, for example.

In one embodiment, the respective sealing is water-tight and/or prevents ingress of dirt.

In one embodiment, the movement of the movable member relative to the exterior operation surface from the initial position towards the operation position, which may be required such that the signal is generatable via the signaling unit, is unidirectional. Complicated multidirectional movements for signal generation may be avoided in this way.

In one embodiment, the user interface member is movable, e.g. axially, from a first position to a second position, for example relative to the housing, such as the housing of the system or the drug delivery device. The first position may be an initial position which the user interface member has relative to the housing, e.g. before a dose setting operation and/or a dose delivery operation is commenced. The second position may be a position which the user interface member assumes when a user force is applied to the user interface member, e.g. a distally directed force, and the user interface member is moved away from the first position, e.g. for the dose delivery operation. The first position and the second position may be axially offset. The movement from the first position to the second position may involve only axial movement.

In one embodiment, the electronic system or the drug delivery device comprises the dose setting and drive mechanism. The first member and/or the second member may be configured to move during the dose setting operation and/or the dose delivery operation relative to the housing of the electronic system or the drug delivery device. The first member may be a dose member or dial member of the dose setting and/or drive mechanism, which is moved to set a dose, e.g. a dial sleeve or a number sleeve. The second member may be a drive member, e.g. a member engaged with a piston rod of the dose setting and/or drive mechanism, or a device user interface member, such as a dose knob and/or injection button. The first member and/or the second member may be movably coupled to or retained in the housing. In the dose setting operation, the first mechanism member and/or the second mechanism member may be displaced axially relative to the housing, for example away from a proximal end of the housing. The distance by which the first member and/or the second member is displaced during the dose setting operation relative to the housing, e.g. axially, may be determined by the size of the set dose. In other words, the drug delivery device may be of the dial extension type, i.e. the device increases its length during the dose setting operation in an amount proportional to the size of the set dose.

In one embodiment, in the dose setting operation and/or in the dose delivery operation, the first member moves, e.g. rotates and/or moves axially, relative to the second member. For example, the first member may rotate relative to the second member during the dose delivery operation, e.g. only during the dose delivery operation. The first member and the second member may both move axially during the dose delivery operation. The first member may rotate relative to the second member and relative to the housing during the dose setting operation and/or the dose delivery operation. The second member may be rotationally locked or guided with respect to the housing during the dose delivery operation, e.g. by a delivery clutch. The first member and the second member may be rotationally locked relative to one another during the dose setting operation. Accordingly, the first member and the second member may rotate relative to the housing in the dose setting operation. During the dose setting operation the first member and the second member may be coupled to one another, e.g. via a coupling interface, e.g. a setting clutch. The coupling interface may rotationally lock the first member and the second member to one another during the dose setting operation. When the coupling interface is engaged or established, the first member and the second member may be rotationally locked with one another, such as by direct engagement of coupling interface features. The first member and the second member may comprise mating coupling interface features. The coupling interface may be released during the dose delivery operation, e.g. by axially displacing the second member relative to the first member. Hence, the second member may be rotationally locked relative to the housing during dose delivery, whereas the first member may rotate relative to the housing during dose delivery. The coupling interface may be released when switching the dose setting and/or drive mechanism from a dose setting configuration into a dose delivery configuration.

This may be achieved when the user interface member is moved from the first position to the second position. In the first position, the mechanism may be in the dose setting configuration. In the second position, the mechanism may be in the dose delivery configuration.

In one embodiment, the first member and the second member rotate relative to one another during only one of the dose setting operation and the dose delivery operation. One of the first member and the second member, e.g. the first member, may rotate relative to the housing during both operations. One of the first member and the second member, e.g. the second member, may rotate relative to the housing during only one of the operations, e.g. during dose setting or during dose delivery.

In one embodiment, the dose setting operation involves a rotational movement of the user interface member relative to a housing, e.g. the housing of the drug delivery device, in a dose setting direction. That is to say both, the movable member and the user interface member body may rotate during dose setting.

In one embodiment for setting a dose in a dose setting operation, the user interface member has to be rotated relative to the housing, e.g. in a dose setting direction. The rotation may be a rotation in whole number multiples of a unit setting increment angle. The unit setting increment may be the smallest dose which can be set to be delivered by the drug delivery device. The unit setting increment angle may be the angle by which the user interface member has to be rotated, e.g. relative to the housing for setting the smallest possible dose.

In one embodiment, the electronic system comprises at least one of, an arbitrarily selected plurality of, or all of the following units or components:
an electrical motion sensing unit. The motion sensing unit will be explained in more detail below.
a communication unit. The communication unit may be provided to establish the communication interface between the electronic system and another device such as an electronic device such as a portable device, e.g. a portable or non-portable computer, a mobile phone or a tablet. The communication unit may be a wireless unit, e.g. an RF communication unit, such as a Bluetooth unit. The communication unit may be provided to transmit dose data from the electronic system to the other device, e.g. information on the amount of drug delivered by the device in a delivery operation.
a memory unit. The memory unit may be provided to store executable program code and/or data on dose information which has been calculated by the electronic system, preferably dose data on the delivered dose or doses. The dose data may be determined via the motion sensing unit. From the memory unit, the data may be retrievable for transmission to another device, e.g. via the communication unit.

In one embodiment, the motion sensing unit is configured to generate one or more electrical motion signals. The motion signal(s) may be suitable to quantify the relative movement between the first member and the second member, e.g. during the dose setting operation or the dose delivery operation, e.g. to obtain dose data, such as the size of the delivered dose. The first member and/or the second member may be members of the electronic system and/or the drug delivery device, e.g. the dose setting and/or drive mechanism as discussed further above. The relative movement may be relative rotational movement. For example, the first member may rotate relative to the second member during dose delivery.

In one embodiment, the electronic system is configured such that the motion sensing unit is switched from the first state into the second state, e.g. by the electronic control unit and/or in response to the provided by the signaling unit in response to the movement of the first portion into the signaling position. In the first state, the motion sensing unit may be not operative to sense movement of the first member relative to the second member. In the second state, the motion sensing unit may be operative. In the second state, the motion sensing unit may have a power consumption which is greater than in the first state. The increase in power consumption of the motion sensing unit may contribute to or define the increased power consumption of the electronic system in the second state.

In one embodiment, the motion sensing unit is configured to operate during the dose delivery operation, preferably only during the dose delivery operation. The motion sensing unit may be configured to monitor the dose delivery operation, e.g. the rotation of the first member relative to the second member. Thus, from the motion signals, positional information on the relative position between the first member and the second member can be gathered. Alternatively or additionally, it is also possible to gather positional information between two members in the dose setting operation. However, in order to calculate dose information or data on the dose delivery during the dose delivery operation, it is advantageous to monitor the movements during the dose delivery operation by the motion sensing unit.

In one embodiment, the electronic control unit or the electronic system is configured to calculate dose information or data utilizing the motion signals generated by the motion sensing unit. As noted previously, the dose information preferably is information on the size of the dose which is delivered in the dose delivery operation.

In one embodiment, the motion sensing unit comprises one or more sensors and/or one or more emitters, e.g. one or more optoelectronic radiation sensors or detectors and/or one or more optoelectronic radiation emitters. The sensors may be configured to generate motion signal(s) in response to movement of the first member relative to the second member. The emitters may excite the sensor signals.

In one embodiment, during the dose setting operation, the dose may be set, e.g. between a minimum settable dose and a maximum settable dose. The dose may be set, preferably in quantities corresponding to whole-number multiples of one unit dosage increment.

In one embodiment, the separation, e.g. the axial separation, between the first position and the second position of the user interface member, e.g. relative to the housing, is determined by, for example equal to, a switching distance, e.g. a clutch release distance. The switching distance may be the distance by which the second member of the dose setting and drive mechanism has to be moved relative to the first member of the dose setting and drive mechanism in order to switch the dose setting and drive mechanism from a dose setting configuration of the mechanism to a dose delivery configuration of the mechanism. In the first position, the dose setting and drive mechanism may be in the dose setting configuration. In the second position, the dose setting and drive mechanism may be in the dose delivery configuration. In the dose setting configuration or the first position, for example, the members of the dose setting and drive mechanism may be rotationally locked as has been discussed further above. In the dose delivery configuration or the second position, relative rotation is allowed, e.g. the first member may rotate relative to the second member and the housing during dose delivery. During the dose delivery operation, the second member may be rotationally locked relative to the housing.

In one embodiment, the, e.g. axial, separation between the first position and the second position is greater than or equal to the distance by which the second member has to be moved, e.g. axially, relative to the first member in order to release the rotational lock. Specifically, during the movement of the user interface member from the first position to the second position, the rotational lock may be released by displacing the second member axially, e.g. distally, relative to the first member. The distance to release the rotational lock may correspond to, or be greater than the switching distance. The signal may be provided and/or the electronic system may be switched to the second state immediately when the member is moved from the first position into the second position.

In one embodiment, the electronic system comprises a power supply, e.g. a rechargeable or non-rechargeable battery.

In one embodiment, in the second state, the electronic system is configured to gather information or data related to the size of the currently dispensed or delivered dose during the delivery operation, e.g. via the motion sensing unit. Consequently, the motion sensing unit may be configured to contribute to retrieve dose data on the dose delivered in the delivery operation, e.g. the currently delivered dose during the dose delivery operation.

In one embodiment, in the second state, the electronic system is configured to store dose data in a dose memory or memory unit of the electronic system. The memory may be transitory or non-transitory. The dose data is expediently derived using measurements or signals of the motion sensing unit.

In one embodiment, in the second state, the electronic system is configured to transmit dose data, e.g. dose data retrieved from the memory, by means of the communication unit to another device or system such as a computing device, e.g. a mobile phone or a portable or non-portable computing unit.

In one embodiment, the electronic system comprises one user interface member, e.g. one integral member, for the dose setting operation and the dose delivery operation or two different user interface members, where one of these members is the user interface member for dose setting and the other one is the user interface member for dose delivery. The two different members are expediently movable relative to one another, e.g. to switch between a dose setting configuration and a dose delivery configuration. If one interface member is used for dose setting and dose delivery, this interface member may have the setting surface and the delivery surface, which, preferably, are not movable relative to one another, especially not for or during dose delivery and/or not for or during dose setting. If two different user-interface members are used, the setting surface and the delivery surface may be on different members and movable relative to one another for or during dose delivery and/or for or during dose setting.

In one embodiment, the electronic system comprises a timer unit. The timer unit may be configured to deactivate the motion sensing unit and/or other electrically powered units of the electronic system after a predetermined time period has elapsed and, preferably when in this time period no motion signal is generated. The timer unit may trigger or cause the electronic system to be switched from the second state back to the first state. In other words, the electronic system may be configured to switch from the second state back to the first state, preferably when for a predetermined time no motion signal is generated and/or received by the electronic control unit.

In one embodiment, the user interface member is a dose setting and/or an injection button of or for the drug delivery device.

In one embodiment, the electronic system comprises a feedback unit. The feedback unit may be configured to generate a feedback perceivable by the user. The feedback may enable the user to determine whether the system is in the first state or in the second state. Preferably, in the first state, there is no perceivable feedback provided and the feedback is indicative for the second state. The feedback may be a feedback signal, such as an optical signal, for example. The feedback signal may be provided by a light source, such as a light-emitting diode. The light source may operate in a pulsed or flashing manner for providing the feedback.

In one embodiment, the device is a manually driven device, e.g. user driven.

In one embodiment, the drug delivery device comprises a reservoir retainer for retaining a reservoir with drug, e.g. a cartridge, and/or the device comprises the reservoir with drug. The reservoir may comprise drug sufficient for a plurality of, preferably user-settable, doses to be delivered by the drug delivery device.

In one embodiment, the drug delivery device is a pen-type device.

In one embodiment, the electronic system is configured as a, preferably reusable, add-on for a drug delivery device unit. The system may be configured to be attached to the drug delivery device unit. That is to say, the electronic system may be configured to be used with a plurality of drug delivery device units. The respective drug delivery device unit may be a disposable drug delivery device unit and/or the respective drug delivery device unit may be fully operational for performing dose setting operations and dose delivery operations. The drug delivery device unit may comprise the reservoir.

In one embodiment, the power supply is non-replaceable.

In one embodiment, a kit for a drug delivery device comprises the drug delivery device unit and the electronic system. The system may be attachable to the device unit to form the drug delivery device. Features disclosed above and below for the drug delivery device, especially the ones that are not directly related to the electronic system, should also apply for the drug delivery device unit and vice versa.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end, where a needle unit is or is to be mounted to the device, for example.

In a particularly advantageous embodiment, an electronic system for a drug delivery device comprises:
at least one user interface member configured to be manipulated by a user for performing a dose operation, e.g. a dose setting operation to set a dose of drug to be delivered by the drug delivery device and/or a dose delivery operation for delivering a set dose,
an electronic control unit, the electronic control unit being configured to control operation of the electronic system, the electronic system having a first state and a second state, wherein the electronic system has an increased electrical power consumption in the second state as compared to the first state, wherein
the user interface member comprises an exterior operation surface which is arranged to be touched by the user for the dose operation, wherein
the user interface member comprises a user proximity detection unit, wherein the user proximity detection unit is configured to generate an electrical signal when the user is close to the exterior operation surface or touches the exterior operation surface, wherein
the user proximity detection unit comprises a movable member, wherein the movable member is arranged to be moved by the user away from an initial position relative to the exterior operation surface towards an operation position before the user reaches the exterior operation surface, wherein
the user proximity detection unit further comprises an electrical signaling unit, wherein the user proximity detection unit is configured to provide the electrical signal when the movable member has been moved away from the initial position, e.g. when the movable member is in the operation position or during the movement away from the initial position towards the operation position, and wherein
the electronic system is configured such that the electronic control unit switches the electronic system from the first state into the second state in response to the electrical signal.

Features, which are disclosed in conjunction with different aspects and embodiments may be combined with one another even if such a combination is not explicitly discussed above or below. Further aspects, embodiments and advantages will become apparent from the following description of the exemplary embodiments in conjunction with the figures.

### BRIEF DESCRIPTION OF THE FIGURES

FIG. 1 illustrates an embodiment of a drug delivery device.

FIG. 2 illustrates schematically an electronic system for a drug delivery device, e.g. the one in FIG. 1.

FIG. 3 illustrates schematically an embodiment of an electronic system for a drug delivery device, e.g. the one in FIG. 1.

FIGS. 4A to 4C illustrate schematically an embodiment of the electronic system.

FIGS. 5A to 5C illustrate schematically an embodiment of the electronic system.

FIGS. 6A to 6C illustrate schematically an embodiment of the electronic system.

FIGS. 7A to 7C illustrate schematically an embodiment of the electronic system.

FIGS. 8A to 8C illustrate schematically an embodiment of the electronic system.

FIG. 9 schematically illustrates schematically an embodiment of the electronic system.

### DETAILED DESCRIPTION

In the figures, identical features, features of the same kind or identically or similarly acting features may be provided with the same reference numerals.

In the following, some concepts will be described with reference to an insulin injection device. The systems described herein may be implemented in this device or used as an add-on module to the device. The present disclosure is however not limited to such an application and may equally well be used for or in injection devices that are configured to eject other medicaments or drug delivery devices in general, preferably pen-type devices and/or injection devices.

In the following, embodiments are provided in relation to injection devices, in particular to variable dose injection devices, which record and/or track data on doses delivered thereby. These data may include the size of the selected dose and/or the size of the actually delivered dose, the time and date of administration, the duration of the administration and the like. Features described herein may include power management techniques (e.g. to facilitate small batteries and/or to enable efficient power usage).

Certain embodiments in this document are illustrated with respect to an injection device where an injection button and grip (dose setting member or dose setter) are combined e.g. similar to Sanofi's ALLSTAR® device. The injection button may provide the user interface member for initiating and/or performing a dose delivery operation of the drug delivery device. The grip or knob may provide the user interface member for initiating and/or performing a dose setting operation. The devices may be of the dial extension type, i.e. their length increases during dose setting. Other injection devices with the same kinematical behavior of the dial extension and button during dose setting and dose expelling operational mode are known as, for example, the Kwikpen® or Savvio® device marketed by Eli Lilly and the FlexPen®, FlexTouch®, or Novopen® device marketed by Novo Nordisk. An application of the general principles to these devices therefore appears straightforward and further explanations will be omitted. However, the general principles of the present disclosure are not limited to that kinematical behavior.

Certain other embodiments may be conceived for application to injection devices where there are separate injection button and grip components/dose setting members e.g. Sanofi's SoloSTAR®. Thus, the present disclosure also relates to systems with two separate user interface members, one for the dose setting operation and one for the dose delivery operation. In order to switch between a dose setting configuration of the device and a dose delivery configuration, the user interface member for dose delivery may be moved relative to the user interface member for dose setting. If one user interface member is provided, the user interface member may be moved distally relative to a housing. In the course of the respective movement, a clutch between two members of the dose setting and drive mechanism of the device changes its state, e.g. from engaged to released or vice versa. When the clutch, e.g. formed by sets of meshing teeth on the two members, is engaged, the two members may be rotationally locked to one another and when the clutch is disengaged or released, one of the members may be permitted to rotate relative to the other one of the two members. One of the members may be a drive member or drive sleeve which engages a piston rod of the dose setting and drive mechanism. The drive sleeve may be designed to rotate relative to the housing during dose setting and may be rotationally locked relative to the housing during dose delivery. The engagement between drive sleeve and piston rod may be a threaded engagement. Thus, as the drive sleeve cannot rotate during dose delivery, axial movement of the drive sleeve relative to the housing will cause the piston rod to rotate. This rotation may be converted into axial displacement of the piston rod during the delivery operation by a threaded coupling between piston rod and housing.

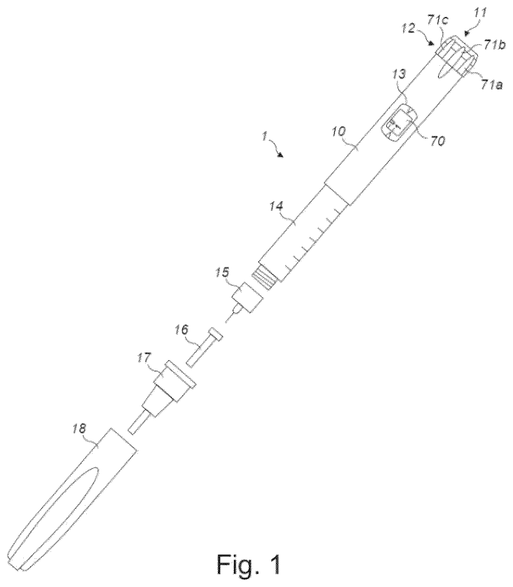

The injection device 1 of FIG. 1 is an injection pen that comprises a housing 10 and contains a container 14, e.g. an insulin container, or a receptacle for such a container. The container may contain a drug, e.g. insulin. The container may be a cartridge or a receptacle for a cartridge which may contain the cartridge or be configured to receive the cartridge. A needle 15 can be affixed to the container or the receptacle. The container may be a cartridge and the receptacle may be a cartridge holder. The needle is protected by an inner needle cap 16 and either an outer needle cap 17 or another cap 18. An insulin dose to be ejected from injection device 1 can be set, programmed, or ‘dialed in’ by turning a dosage knob 12, and a currently programmed or set dose is then displayed via dosage window 13, for instance in multiples of units. The units may be determined by the dose setting mechanism which may permit relative rotation of the knob 12 to the housing 10 only in whole-number multiples of one unit setting increment, which may define one dosage increment. This may be achieved by an appropriate ratchet system, for example. The indicia displayed in the window may be provided on a number sleeve or dial sleeve 70. For example, where the injection device 1 is configured to administer human insulin, the dosage may be displayed in so-called International Units (IU), wherein one IU is the biological equivalent of about 45.5 micrograms of pure crystalline insulin (1/22 mg). Other units may be employed in injection devices for delivering analogue insulin or other medicaments. It should be noted that the selected dose may equally well be displayed differently than as shown in the dosage window 13 in FIG. 1.

The dosage window 13 may be in the form of an aperture in the housing 10 or a transparent separate component inserted into an aperture of the housing, where the separate component may incorporate a magnifying lens. The dosage window 13 permits a user to view a limited portion of a dial sleeve 70 that is configured to move when the dosage knob 12 is turned, to provide a visual indication of a currently programmed dose. The dosage knob 12 is rotated on a helical path with respect to the housing 10 when turned during programming.

In this example, the dosage knob 12 includes one or more formations 71a, 71b, 71c to facilitate attachment of a data collection device or electronic system. An electronic system which may be attachable to the user interface member (knob 12 and/or button 11) or, in general, to elements or members of a dose setting and drive mechanism of the drug delivery device 1 will be described in more detail below. The electronic system may be provided within the user interface member, for example. The electronic system which will be described in more detail below can also be configured as an add-on for a drug delivery device unit, e.g. the unit shown in FIG. 1. In this case, the electronic is preferably configured to be used with a plurality of drug delivery device units. The respective drug delivery device unit is expediently disposable.

The injection device 1 may be configured so that turning the dosage knob 12 causes a mechanical click sound to provide acoustical feedback to a user. In this embodiment, the dosage knob or dose button 12 also acts as an injection button 11. When needle 15 is stuck into a skin portion of a patient, and then dosage knob 12/injection button 11 is pushed in an axial direction, the insulin dose displayed in display or dosage window 13 will be ejected from injection device 1. When the needle 15 of injection device 1 remains for a certain time in the skin portion after the dosage knob 12 is pushed home, the dose is injected into the patient's body. Ejection of the insulin dose may also cause a mechanical click sound, which is however different from the sounds produced when rotating the dosage knob 12 during dialing of the dose.

In this embodiment, during delivery of the insulin dose, the dosage knob 12 is returned to its initial position in an axial movement, without rotation, while the dial sleeve 70 or number sleeve 70 is rotated to return to its initial position, e.g. to display a dose of zero units. As noted already, the disclosure is not restricted to insulin but should encompass all drugs in the drug container 14, especially liquid drugs or drug formulations.

Injection device 1 may be used for several injection processes until either the insulin container 14 is empty or the expiration date of the medicament in the injection device 1 (e.g. 28 days after the first use) is reached.

Furthermore, before using injection device 1 for the first time, it may be necessary to perform a so-called “prime shot” to ensure fluid is flowing correctly from insulin container 14 and needle 15, for instance by selecting two units of insulin and pressing dosage knob 12 while holding injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user.

As explained above, the dosage knob 12 also functions as an injection button 11 so that the same component is used for dialling/setting the dose and dispensing/delivering the dose. Again, we note that a configuration with two different user interface members which, preferably only in a limited fashion, are movable relative to one another is also possible. The following discussion will, however, focus on a single user interface member which provides dose setting and dose delivery functionality. In other words, a setting surface of the member which is touched by the user for the dose setting operation and a dose delivery surface which is touched by the user for the dose delivery operation are immovably connected. Alternatively, they may be movable relative to one another, in case different user interface members are used. During the respective operation, the user interface member is preferably moved relative to the body or housing of the device. During dose setting the user interface member is moved proximally and/or rotates relative to the housing. During dose delivery, the user interface member moves axially, e.g. distally, preferably without rotating relative to the housing or body.

In the following, a general setup for an electronic system for a drug delivery device is disclosed.

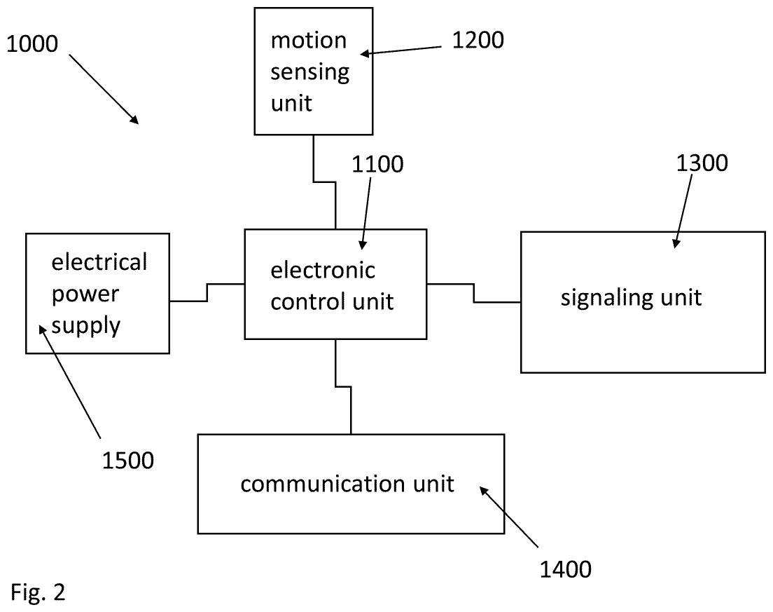

FIG. 2 illustrates a general configuration of elements of an electronic system 1000 which can be used in or for a drug delivery device, for example the device or device unit 1 discussed further above or in or for different devices.

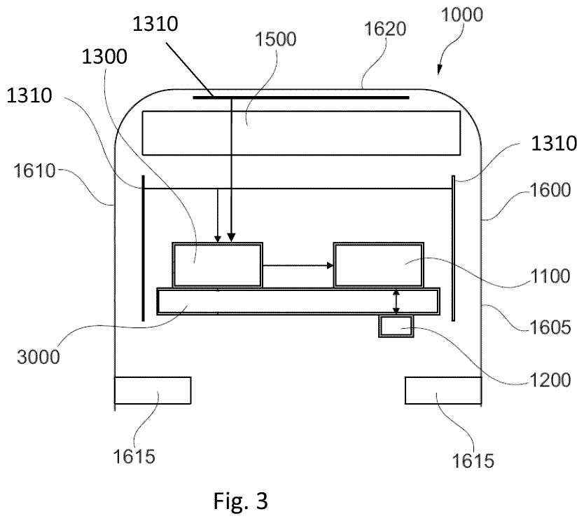

The electronic system 1000 comprises an electronic control unit 1100. The control unit may comprise a processor, e.g. a microcontroller or an ASIC. Also, the control unit 1100 may comprise one, or a plurality of memory units, such as a program memory and/or a main memory. The program memory may be designed to store program code which when carried out by the system controls operation of the system and/or the electronic control unit. The control unit 1100 is expediently designed to control operation of the electronic system 1000. The control unit 1100 may communicate via wired interfaces or wireless interfaces with further units of the electronic system 1000. The control unit 1100 may transmit signals containing commands and/or data to the respective unit and/or receive signals and/or data from the respective unit. The connections between the units and the electronic control unit 1100 are symbolized by the lines in FIG. 2. However, there also may be connections between the units, which are not illustrated explicitly. The control unit 1100 may be arranged on a conductor carrier, e.g. a (printed) circuit board (see reference 3000 in FIG. 3). The other unit(s) of the electronic system may comprise one or more components which are arranged on the conductor carrier as well or on an additional conductor carrier, as the case may be.

Electronic system 1000 further comprises an electrical motion sensing unit 1200. The motion sensing unit 1200 may comprise one sensor e.g. only one sensor, or a plurality of sensors. The motion sensing unit is expediently designed to generate motion signals, such as electrical signals, which are indicative for movement of one member of the electronic system or the drug delivery device relative to another member—e.g. movement of the dial sleeve or number sleeve relative to the drive sleeve or button/knob in the device discussed further above—, where the sensor may be fixedly connected to one of the members, e.g. the knob or button. The relative movement expediently occurs during the dose delivery operation. The respective sensor may be an optoelectronic sensor. The optoelectronic sensor may sense radiation emerging from a member moving relative to the sensor and impinging on the sensor to excite sensor signals or motion signals in the sensor, e.g. an optical encoder component. The radiation may be radiation reflected by the member and impinging on the member from a radiation source, such as an optoelectronic radiation source, e.g. an LED. The radiation source may be an IR source (IR-LED, an InfraRed Light Emitting Diode). The radiation source may be part of the sensor arrangement comprising the at least one sensor. One possible embodiment of the sensor is an IR-sensor which is configured to detect infrared light. The light source and the sensor may be arranged on the same component or member. The general functionality of optoelectronic sensor arrangements suitable for the electronic system discussed herein is disclosed in WO 2019/101962 A1, where the entire disclosure content is explicitly incorporated herein by reference for all purposes, especially as regards the different sensor arrangements and configurations. However, it should be noted that other sensor arrangements, e.g. using magnetic sensors, could be employed as well. In a motion sensing unit which has an electrically operated sensor and/or an electrically operated source for stimulating the sensor—such as a radiation emitter and an associated sensor—the power consumption may be particularly high and, hence, appropriate power management of electrical power available for powering the system may have a particular impact. The motion sensing unit 1200 may be designed to detect and preferably measure or quantify relative movement of one member of a dose setting and drive mechanism of or for the drug delivery device relative to another member of the dose setting and drive mechanism or relative to the housing 10 during a dose delivery operation. For example, the motion sensing unit may measure or detect relative rotational movement of two movable members of the dose setting and drive mechanism with respect to one another. Based on movement data received from or calculated from the signals of the unit 1200, the electronic system, e.g. the control unit, may calculate dose data, e.g. data on the currently delivered dose. The motion sensing unit 1200 is expediently configured to quantify the relative movement between a first member and a second member of the electronic system or the drug delivery device. The relative movement may be indicative for the delivered dose. The relative movement may be relative rotational movement. For example, the first member may rotate relative to the second member, such as during dose delivery. The motion sensing unit is expediently suitable to quantify the relative movement in whole-number multiples of one unit setting increment angle. The unit setting increment may be or may be defined by an angle greater than or equal to one of the following values: 5°, 10°. The unit setting increment may be or may be defined by an angle less than or equal to one of the following values: 25°, 20°. The unit setting increment may be between 5° and 25°, for example. The unit setting increment may correspond to a relative rotation of 15°, for example. The unit setting increment angle may be the rotation required to set the smallest settable dose to be delivered by the device. The increment may be defined by a ratchet system for example. As has been explained above, the amount or distance of the relative (rotational) movement determined by the motion sensing unit between the first and second members is characteristic for the currently set dose in a dose setting operation or for the currently dispensed dose in a dose delivery operation. The size of the dose delivered may be determined by or correspond to the distance by which a piston rod of the dose setting and drive mechanism is displaced distally relative to the housing during the dose delivery operation.

The electronic system 1000 further comprises a signaling unit 1300. The signaling unit may be associated with the user interface member or members (knob 12 or button 11 in the device discussed above). Via the signaling unit 1300 the manipulation of the member for setting and/or for delivering a dose may be detected or indicated. The signaling unit is configured to generate an electrical signal which is indicative that an exterior operation surface of the user interface member, e.g. a body thereof, is being touched or that the user is in proximity to this surface. The user interface member may have a setting surface which is arranged to be touched by the user for performing the dose setting operation and/or a delivery surface which is arranged to be touched by the user for performing the dose delivery operation. The setting surface may face in the radial direction and the delivery surface may face in the axial, e.g. proximal, direction. Signal generation may require movement of at least a portion of the user interface member, e.g. relative to another portion of the user interface member and/or relative to the housing 10. The signaling unit may be part of a user proximity detection unit which is configured to provide an electrical signal, when, preferably only when, the user touches an exterior operation surface of the user interface member, e.g. the setting surface for a setting operation and/or the delivery surface for a delivery operation. The user proximity detection unit further comprises a movable member which is accessible on that exterior surface relative to which proximity of the user should be detected and expediently proud of that surface. Examples for the movable member are set forth further below. The movable member and the signaling unit are expediently adjusted such that the signaling unit can provide a signal only if the movable member has been displaced away from an initial position relative to the exterior operation surface (e.g. protruding from the surface) towards the surface, e.g. towards or into an operation position, i.e. a position which it assumes during the operation performed by the manipulation of the interface member via the exterior operation surface. Expediently, the movable member is arranged such that it has to be moved before the user can touch the exterior operation surface and/or before a signal can be provided by the signaling unit. Hence, if a manipulation of the user interface member is performed without moving the movable member towards or into the operation position a signal may not be generated by the signaling unit. The movable member may have to be in the operation position for the signal to be generated or the signal may be generated before the operation position is reached. The movable member may stay in the operation position during the entire manipulation performed by the user for conducting the operation, e.g. dose setting or delivery. Embodiments with the movable member will be described in more detail below. The element generating or causing generation of the signal may be an electrical sensor or switch, such as a micro switch, for example. The signals generated by the signaling unit in response to manipulations may allow to distinguish between different surfaces of the user interface member which are manipulated by the user. In this case, a plurality of switches may be provided, one for the setting surface and one for the delivery surface. The signaling unit is expediently configured such that the electrical signal(s) it is configured to generate responsive to a manipulation allows gathering information on what operation is currently being performed or is intended to be performed, e.g. a dose setting operation or a dose delivery operation. The signal generated by the signaling unit 1300 may be an activation prompt signal or use signal. The signaling unit 1300 is operatively connected to the electronic control unit 1100, for example. The signal provided by the signaling unit may be received and/or processed by the electronic control unit 1100.

The electronic system 1000 further comprises a communication unit 1400, e.g. an RF, WiFi and/or Bluetooth unit. The communication unit may be provided as a communication interface between the system or the drug delivery device and an external device, such as other electronic devices, e.g. mobile phones, personal computers, laptops and so on. For example, dose data may be transmitted by the communication unit to the external device and/or synchronized with the device. The dose data may be used for a dose log or dose history established in the external device. The communication unit may be provided for wireless communication.

Occurrence of the signal generated by the signaling unit 1300 may cause the electronic control unit 1100 to switch the electronic system 1000 from a first state or rest state (e.g. the state the system has when it is not needed, e.g. a dormant state, where the rest state is optimized in terms of low power consumption) to a second state of higher power consumption, e.g. by activating the motion sensing unit 1200 and/or the communication unit 1400. For this purpose, the control unit 1100 may send an activation signal to the respective unit. In the second state, the motion sensing unit and/or the communication unit may be operable. In the first state, the motion sensing unit and/or the communication unit, preferably, cannot be operated. In this way, the functionality of the electrically operated units may be available when needed. The power consumption required in the first state for the signaling unit being operational advantageously is smaller than the power consumption when the communication unit and/or the motion sensing unit are/is operable. The activation prompt or use signal may be generated responsive to a manipulation of a portion of the user interface member, e.g. the movable member. The manipulation may involve only unidirectional movement of the movable member, e.g. distal movement for the dose delivery operation. The manipulation may involve only linear and/or axial movement of the movable member relative to the exterior operation surface.

The electronic system 1000 further comprises an electrical power supply 1500, such as a rechargeable or non-rechargeable battery. The power supply 1500 may provide electrical power to the respective units of the electronic system.

In one embodiment, the power consumption, in particular the maximum power consumption, of the electronic system in the first state, e.g. prior to generation of the use or activation prompt signal, may be less than or equal to one of the following values: 300 nA, 250 nA, 200 nA (nA: nanoampere). Alternatively or additionally, in the second state of the electronic system, the power consumption, in particular the minimum power consumption, may be greater than or equal to one of the following values: 0.5 mA, 0.6 mA, 0.8 mA (mA: milliampere). The difference can result from the power consumption of the motion sensing unit 1200 and/or of the communication unit 1400 which may be active or operable in the second state and switched off or in a sleep state in the first state of the electronic system 1000.

In one embodiment, the power consumption P2, e.g. the minimum or maximum power consumption, in the second state may be greater than or equal to at least one of the following values: 2*P1, 3*P1, 4*P1, 5*P1, 10*P1, 20*P1, 30*P1, 40*P1, 50*P1, 100*P1, 500*P1, 1000*P1, 2000*P1, 5000*P1, 10000*P1 where P1 is the power consumption in the first state. In the second state, the motion sensing unit may be active and/or the communication unit may be active, e.g. for wireless communication.

When the system is in the first state, e.g. with neither the motion sensing unit being active nor the communication unit, the current consumption may be 200 nA. When (only) the motion sensing unit is active, the power consumption may be 0.85 mA. When the communication unit is active, e.g. in addition to the motion sensing unit or only the communication unit, the power consumption may be 1.85 mA.

Although not explicitly depicted, the electronic system preferably comprises a, e.g. permanent and/or non-volatile, storage or memory unit, which may store data related to the operation of the drug delivery device such as dose (history) data, for example.

In one embodiment, the electronic control unit 1100 is configured to reduce the power consumption of the respective unit, i.e. to switch the unit back to the first state. This is suitable, for example, if an event which is relevant for that unit, e.g. a motion sensing event (motion signal) for the motion sensing unit, has not occurred in a predetermined time interval after the unit has been switched from the first state into the second state and/or after the use signal has been generated. The monitoring of the time interval may be achieved by a timer unit which is operatively connected to the electronic control unit (not explicitly shown). In case, after the use or activation prompt signal, there is no signal generated by the motion sensing unit within the predetermined time interval, the entire system may be switched to the first state again. This time interval may be greater than or equal to one of the following values 5 s, 10 s, 15 s, 20 s, 25 s, s. Alternatively or additionally the time interval may be less than or equal to one of the following values: 180 s, 150 s, 120 s, 90 s, 80 s, 70 s, 60 s, 50 s, 45 s, 40 s, 35 s, 30 s. The time interval may be between 5 and 180 seconds, e.g. 30 s or 180 s. The entire system may be switched back to the first state in case no motion signal is generated within the predetermined time interval. The predetermined time interval is expediently constant.

In one embodiment, the electronic system comprises a feedback unit (not explicitly shown). The feedback unit is configured to generate a feedback perceivable by the user. The feedback can enable the user to determine whether the system is in the first state or in the second state. Preferably, in the first state, there is no perceivable feedback provided and/or the feedback is indicative for the second state. The feedback may be a feedback signal, such as an optical signal, for example. The feedback signal may be provided by a light source, such as a light-emitting diode. The light source may operate in a pulsed or flashing manner for providing the feedback.

The respective unit which has been described above may be integrated into the user interface member of the electronic system which is discussed in further detail below in conjunction with various embodiments.

It goes without saying that the electronic system 1000 may comprise further electronic units other than the ones shown such as other sensing units, which sense or detect different quantities or events than the relative movements which the motion sensing unit detects.

In the following some more detailed embodiments of the electronic system are described. It should be noted that features which have been discussed above do also apply for these embodiments.

FIG. 3 schematically illustrates an embodiment of an electronic system 1000. The system 1000 comprises a user interface member 1600. The user interface member is designed to be operated during a dose setting operation and/or a dose delivery operation by the user. The user interface member 1600 has different exterior operation surfaces. The operation surfaces may be defined by exterior surfaces which are accessible from the exterior of a user interface member housing or body 1605, preferably when the user interface member is connected to a drug delivery device unit or integrated into a device such as the unit or the device discussed in conjunction with FIG. 1. The user interface member 1600 has a setting surface 1610 which is arranged to be gripped by the user for dose setting, e.g. with two fingers such as the index finger and the thumb. The setting surface is a radially facing surface, which, preferably circumferentially, delimits the user interface member 1600 with respect to the exterior. The user interface member 1600 also has a delivery surface 1620. The delivery surface is arranged to be contacted, e.g. pressed and/or moved distally, by the user for dose delivery. The delivery surface 1620 is an axially oriented surface, e.g. a proximally facing surface. As noted above, embodiments of the disclosure can employ different user interface members for setting and delivery.

Within the user interface member 1600, e.g. within an interior hollow defined by the user interface member body 1605, some additional elements or units of the electronic system are housed. Specifically, the electronic system comprises the electronic control unit 1100. The system also comprises a conductor carrier 3000, e.g. a circuit board such as a printed circuit board. Conductors on the conductor carrier may conductively connect the electronic control unit to further electrical or electronic units or members of the system. The carrier may be connected to the user interface member body 1605 relative to the body 1605, e.g. axially and/or rotationally fixed relative to body 1605.

The electronic system 1000 comprises the signaling unit 1300. The electronic control unit and/or the signaling unit or at least a component thereof is arranged on the conductor carrier, e.g. mounted to the carrier. In the depicted embodiment, the signaling unit has at least one sensor or switch or a plurality of sensors or switches 1310. In the depicted embodiment, at least one switch 1310 is associated with the setting surface 1610. Alternatively or additionally, at least one switch 1310 is associated with the delivery surface 1620 (the switches are only schematically illustrated in this embodiment). The respective switch is expediently configured to generate an electrical use signal or switch signal (only) when the user touches the setting surface for performing the dose setting operation (the sensor or switch is expediently associated with the setting surface) or the delivery surface for the dose delivery operation (the sensor or switch is expediently associated with the delivery surface). The signal in the present disclosure may consist of just one signal pulse, e.g. a voltage or current pulse, or just one change in an electrical characteristic, e.g. a change in voltage or current, caused by triggering the signaling unit 1300, e.g. the switch 1310. For the dose setting operation, the user interface member 1600 may be rotated relative to the housing 10. For the dose delivery operation, the user interface member can be moved axially towards the housing, e.g. to switch the clutch such as from the state where the dial sleeve and the drive member are rotationally locked for dose setting to a state where relative rotation is allowed for dose delivery. The user interface member is preferably biased, e.g. by a clutch spring (not shown) and/or relative to the housing 10, to the position it has for dose setting which may be proximally offset to the one for dose delivery by the clutch switching distance. The clutch switching distance (the distance the user interface member has to be moved in order to switch the clutch) is for example greater than or equal to 1.5 mm.

The electrical signal generated by the signaling unit—use or activation prompt signal—may directly trigger the electronic control unit 1100 to switch the system from the first state to the second state. The movement, which triggers generation of the signal, is expediently unidirectional, i.e. only movement in one direction is required to switch the system to the second state. In this way complicated manipulations of the user interface member 1600 or elements thereof such as the movable member discussed further below for switching the system to the second state can be avoided.

The system furthermore comprises the motion sensing unit 1200 which is only schematically represented and, preferably, comprises one or more optoelectronic sensors and/one or more associated radiation emitters, e.g. IR sensors and IR emitters. The motion sensing unit may be bidirectionally conductively connected to the electronic control unit 1100 as hinted by the double arrow. One direction may be the one where the activation signal is transmitted from the electronic control unit to the motion sensing unit. In the other direction, motion signals may be sent from the motion sensing unit to the control unit, which may process the signals further, e.g. to calculate dose information or data. The motion sensing unit 1200 may be arranged on that side of the conductor carrier 3000 which faces away from the control unit 1100 or the delivery surface 1620.

Further, the system 1000 comprises the power supply 1500, e.g. a battery, such as a coin cell. The power supply may be configured to provide a total charge of approx. 25-500 mAh at a voltage of approx. 1.4-3V. This may be achieved or assisted by stacking multiple coin cells, for example. The power supply 1500 is conductively connected or connectable to the other components of the electronic system, which require electrical power for operating. The conductive connection is not explicitly illustrated in FIG. 3. A metal pressing may be provided for connecting the power supply 1500 to the conductor carrier 3000 which may distribute the power to further elements via conductors on the carrier (not explicitly shown). The power supply may, however, be arranged so as to extend along one main surface of the conductor carrier 3000 as depicted. The power supply, in the depicted embodiment, is arranged between the conductor carrier 3000 and the delivery surface 1620. This facilitates a compact formation of the user interface member 1600.

A radial width or diameter of the user interface member 1600 as seen from the exterior of the member, e.g. in top view onto the delivery surface, may be less than or equal to one of the following values: 2 cm, 1.5 cm. Alternatively or additionally, the radial width or diameter of the user interface member may be greater than or equal to one of the following values: 0.5 cm, 0.7 cm. The radial extension may be determined relative to the rotation axis of the user interface member during dose setting or relative to the main longitudinal axis of the user interface member, which axes may coincide. The length or axial extension of the user interface member 1600 may be less than or equal to one of the following values: 2.5 cm, 2 cm, 1.5 cm.

Alternatively or additionally, the length or axial extension of the user interface member 1600 may be greater than or equal to one of the following values: 0.5 cm, 0.7 cm.

Electronic system 1000 is configured to be connected, preferably releasably, to a drug delivery device unit as an add-on unit or module. The drug delivery device unit may be electronic free. Accordingly, all electronics and/or all electrically conducting or conductive components may be provided in the electronic system. The drug delivery device unit may be disposable. That is to say, the unit can be disposed of after a reservoir of the unit has been emptied using the drug delivery device comprising the unit and the system 1000. The electronic system 1000 could be reused for another drug delivery device unit. The drug delivery device unit is preferably configured as fully functional on its own, i.e. it could be operated for setting a dose to be delivered and deliver the set dose. One exemplary unit is the one depicted in FIG. 1. The electronic system may be a pure add-on to an, otherwise, fully functional unit. Alternatively, a drug delivery device may comprise the electronic system as an integral part, i.e. a part which is disposed of together with the remainder of the device and/or necessary such that the device can be operated for setting and delivering a dose of drug, e.g. because without the electronic system the drug delivery device unit would lack a surface accessible for the user for conducting a dose setting operation or a dose delivery operation. For a connection to the drug delivery device unit, the electronic system 1000 may comprise one or more connection features 1615, e.g. snap features. The respective connection feature is arranged in a distal portion of the user interface member 1600, e.g. in the interior of the member.

The system 1000 is expediently configured to be mechanically connected, either permanently or removably/releasably, to a member of the drug delivery device unit such as a member of the dose setting and drive mechanism, e.g. to the drive sleeve or the dose knob and/or the injection button of the unit discussed in conjunction with FIG. 1. The system, e.g. via the user interface member body 1605, may be rotationally and axially locked to the member of the drug delivery device unit. The member to which the system is connected may be movable relative to the housing 10 during dose setting and/or dose delivery, e.g. rotationally and/or axially during setting and, e.g. only, axially during delivery. The member can engage the piston rod, e.g. threadedly. The dose knob and the drive sleeve of the unit in FIG. 1 can be formed integral or act as a single member during dose setting and dose delivery. During dose setting, the drive sleeve may be selectively rotationally locked to a dial sleeve of the dose setting and drive mechanism such that the dial sleeve and the drive sleeve co-rotate during dose setting, e.g. by a clutch, and the dial sleeve rotates relative to the drive sleeve during dose delivery. The dial sleeve may be the number sleeve. The relative rotation between dial sleeve and drive sleeve during dose delivery may be measured by the motion sensing unit. However, it will be readily apparent to those skilled in the art that the disclosed concepts will also work with dose setting and drive mechanisms having different ways of operating and/or different configurations.

The following embodiments illustrate implementations of the user proximity detection unit which comprises the signaling unit 1300 and a movable member 1670. In each case, the signaling unit 1300 is configured to provide or to generate the signal, e.g. the use signal or the activation prompt signal. The embodiments rely on moving a movable member relative to the exterior operation surface, particularly the delivery surface, before the operation surface is contacted by the user. The signal may be provided only when the movable member is in an operation position or when the movable member has been moved away from an initial position to the operation position by the user relative to the exterior operation surface. The signal may be generated during the movement of the movable member relative to the exterior operation surface from the initial position into the operation position, e.g. by triggering the switch 1310. The switch and/or the signaling unit may have a fixed position relative to the exterior operation surface in this case. Alternatively, the movable member in the operation position may be moved together with the exterior operation surface in order to trigger signal generation, e.g. by triggering a switch. In this case, the relative position between the signaling unit and the exterior operation surface may be variable, preferably within specified limits. The system is expediently configured such that the signal is generated before the dose setting and drive mechanism is switched from the dose setting configuration into the dose delivery configuration, i.e. before the clutch is released. The user proximity detection unit discussed in the following embodiments uses the delivery surface as exterior operation surface. However, it will be appreciated that according constructions could also be implemented for the setting surface.

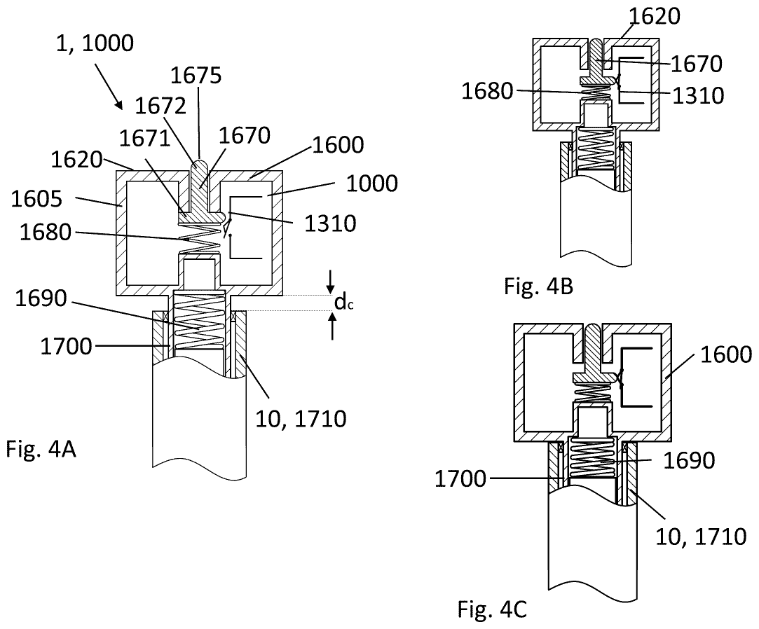

FIGS. 4A through 4C schematically illustrate one embodiment of an electronic system utilizing such a user proximity detection unit with a movable member 1670. FIG. 4A schematically illustrates a sectional view of a proximal section of a drug delivery device 1 or an electronic system 1000 thereof or therefore with the user interface member 1600. In the depicted embodiment, the movable member 1670 protrudes proximally from the delivery surface 1620 of the user interface member 1600 through an opening in the user interface member body 1605 in the initial position depicted in FIG. 4A. The delivery surface is formed by the user interface member body 1605. As noted, the delivery surface 1620 is merely an example for an exterior operation surface and the proposed concepts would also work for the setting surface as exterior operation surface. Hence, referrals to the delivery surface should not be construed as limiting. However, if the electronic system 1000 is designed to operate during the dose delivery operation, waking the system by monitoring or ensuring proximity of the user to the delivery surface is expedient, since that surface needs to be touched for the delivery operation. Having to move the movable member 1670 before the surface is touched ensures that the requirements for signal generation—and accordingly for waking the system by switching it to the second state—are met before or when the user touches the surface, preferably before the exterior operation surface is moved relative to the housing 10 or another component of the device. The movable member 1670 extends through an opening in the user interface member body 1605 from an interior of the user interface member body to the exterior. The protruding portion 1672, in FIG. 4A, is proud of the delivery surface or an enveloping surface of the user interface member body defined by the outer contour of the delivery surface. Thus, a contact surface 1675 of the movable member 1670 is elevated relative to the delivery surface.

When seen in top view onto the delivery surface 1620, a contact surface area (e.g. the proximal surface of the protruding portion) provided by the movable member 1670 is smaller than the surface area of the delivery surface 1620. In other words, the movable member may occupy a surface area which is smaller than the surface area formed by the delivery surface 1620 of the user interface member 1600. The area of the delivery surface preferably is greater than or equal to the combined contact surface area CA formed or defined by the movable member 1670. The area of the delivery surface 1620 may be greater than or equal to one of the following: 3CA, 5CA, 7CA, 10CA, 15CA, 20CA, 30CA, 40CA, 50CA, 75CA, 100CA, 200CA. The combined surface area considers that there may be more than one protruding portion of the movable member accessible on the delivery surface, where the combined contact surface area comprises the sum of all contact surface areas of portions of the movable member accessible on the delivery surface. An according relation may be valid for the combined opening area of the area which is covered by the openings through which the movable member can communicate with the interior of the user interface member body. If the delivery surface is plane and has a circular shape with a diameter of 15 mm and one movable member with a plane circular contact surface of a diameter of 1 mm is provided, the movable member protruding through the delivery surface through an opening of the same diameter as the movable member, the delivery surface is 224 times the combined contact surface area, i.e. the area covered by the movable member. It will be appreciated that other configurations are possible depending on the number of contact surfaces available.

In the first or initial position depicted in FIG. 4A, the movable member 1670 protrudes from the exterior operation surface 1620. The initial position is that position which the movable member has relative to the delivery surface, before the movable member is displaced by the user. The movable member 1670 may, in the initial position, protrude by a distance from the delivery surface 1620 of greater than or equal to one of the following values: 0.1 mm, 0.2 mm, 0.3 mm 0.4 mm, 0.5 mm, 0.6 mm, 0.7 mm, 0.8 mm. 0.9 mm, 1 mm, 1.5 mm. The movable member 1670 may, in the first position, protrude by a distance from the delivery surface of less than or equal to one of the following values: 2 mm, 1.5 mm, 1 mm, 0.9 mm, 0.8 mm, 0.7 mm, 0.6 mm, 0.5 mm, 0.4 mm, 0.3 mm, 0.2 mm, 0.1 mm.

The movable member 1670 is movable relative to the delivery surface 1620 of the user interface member 1600 from the initial position in the distal direction (downwards in FIG. 4A) towards a second or operation position (see FIGS. 4B and 4C). In the operation position, the portion of the movable member which protruded from the delivery surface 1620 in the initial position (this is the portion which provides the user contact surface 1675) is expediently flush (that is to say axially aligned) with the delivery surface or recessed or sub-flush relative to the delivery surface 1620 and/or relative to an enveloping surface of the user interface member body which is defined by the regions of the body 1605 adjoining the opening through which the portion of the movable member projects. In case there are a plurality of portions projecting through openings, every portion may be recessed or sub-flush relative to the adjacent region of the delivery surface. That the portion of the movable member is sub-flush in the operation position may be achieved by dimensioning the opening such that the elasticity of the user's skin on the finger of the user which presses on the movable member is sufficient to move the portion of the movable member below the delivery surface while maintaining contact with the delivery surface to exert a force on that surface, e.g. to drive the dose delivery operation. Thus, in the operation position, the contact surface 1675 may be distally offset from the delivery surface. This is not expressly shown in FIGS. 4B and 4C which show a flush arrangement. However, a sub-flush arrangement can nevertheless be achieved. In the second or operation position, a proximal end of the movable member 1670 and/or its contact surface 1675 may be arranged offset from the delivery surface into the distal direction and/or away from the initial position by a distance which is greater than 0 mm, e.g. greater than 0.05 mm, and less than or equal to one of the following values: 0.5 mm, 0.4 mm, 0.3 mm, 0.2 mm, 0.1 mm.

In the depicted embodiment, the movable member 1670 is generally pin-shaped. The movable member also has one or more radially protruding features 1671 which are arranged to abut features on the user interface member body which prevent the movable member from being removed from the user interface member body. It is, however, noted that other configurations are also possible. Also, the movable number 1670 protrudes from the delivery surface 1620 just in one location in the depicted embodiment. It should be noted that the movable member may protrude from the delivery surface at a plurality of, preferably distinct and/or separate, locations. The movable member 1670 is expediently configured and arranged such that the user contacts the movable member before the user contacts the delivery surface. The system is configured such that the movable member has to be moved into the distal direction away from the first position before the user can contact the delivery surface. The system is further configured such that switch 1310 retained in the user interface member body, e.g. fixed relative to that body 1605, is triggered by the movement of the movable member from the initial position to the operation position and a use signal is provided to the electronic control unit, the system preferably causing the unit to power up the motion sensing unit, for example. The sensor or switch 1310 is illustrated in FIG. 4A schematically. The switch 1310 may be a momentary switch, e.g. an axial micro switch. In the position in FIG. 4B, the switch has been triggered.

The system 1000 is configured such that the movable member 1670 is biased towards the first position. That is to say, when the movable member 1670 is in the second position, the bias tends to move the member into the first position which, accordingly, is the standard position when no force is applied. For this purpose, a biasing member 1680 is provided, in the depicted embodiment a spring, such as a helical compression spring. During the movement of the movable member away from the initial position, the bias may be increased or the member may be loaded. The force exerted onto the biasing member 1680 via the movable member may be reacted by an interior surface of the user interface member body 1605. The force required to move the movable member relative to the exterior operation surfaces from the initial position into the operation position is expediently less than the force which is required to move the user interface member or the delivery surface axially for a dose delivery operation. This facilitates moving the movable member into the operation position before the user interface member is moved. In the operation position, it is expediently still possible to move the movable member further away from the initial position, i.e. distally. The system does not comprise a distal end stop for the movable member and the biasing member 1680 could still be biased or compressed further in the operation position. However, such a (distal) movement of the movable member does not occur in the operation position, as the users skin will be deflected or deformed only up to a certain extent depending on the size and shape of the opening(s) through which the movable member(s) protrude. The size of the opening limits the distance by which the users skin can protrude into the opening.

In the present embodiment, the user interface member 1600/the electronic system 1000 is shown in the state when it is mounted to a drug delivery device unit or integrated in a drug delivery device. The drug delivery device (unit) is symbolized by an element which may be either housing 10 or a member 1710 of the dose setting and drive mechanism of the device (unit). It should be understood, however, that the representation in FIG. 4 is pretty schematic and that the user interface member body may be provided as an add-on to the drug delivery device unit with connection features for a releasable connection. The connection between the drive mechanism and the user interface member is not explicitly shown herein, as the nature of the connection is not important for the general operation of the system which is described here. The connection, however, is configured to transfer dose setting force or torque or delivery force or torque from the respective operation surface to the member 1710.

The electronic system 1000 is expediently configured such that the use signal is generated before and/or such that the system has been switched into the second state before a movement of members of a dose setting and drive mechanism relative to one another occurs which movement may be required for performing the dose delivery operation which results in a distal movement of a piston rod to drive the dispensing operation to dispense medicament from a container. The movement which may be required for the dose delivery operation may be relative rotational movement between the dial sleeve and the drive sleeve of the device discussed further above, for example.

The use signal may be generated before the user interface member body is moved at all via the delivery surface. For example, the use signal may be generated before the user interface member 1600 is moved relative to the housing 10 or member 1710 for initiating the dose delivery operation. Alternatively, the use signal may be generated after movement of the user interface member relative to the housing 10 or member 1710 has been initiated, e.g. movement in the distal direction, but before a clutch interface which couples, e.g. rotationally locks, two members of the dose setting and drive mechanism to one another during the dose setting operation is released for the dose delivery operation. The clutch interface may be formed between at least one member of the dose setting and drive mechanism which is arranged in the chain of force transfer from the delivery surface to a piston rod of the dose setting and drive mechanism and another component, e.g. the housing 10 or another member of the dose setting and drive mechanism. In FIG. 4A the clutch interface may be established. The user interface member may have to be moved by the distance d c relative to the housing 10 or the member 1710 in order to switch the clutch interface, e.g. from established to released, from the situation shown in FIG. 4A. A clutch biasing member 1690, e.g. a spring, such as a helical compression spring, is provided which tends to maintain the clutch interface in one state, e.g. established or released. In the depicted embodiment the two members which are connected by the clutch interface are represented by a drive mechanism member 1700 of the drug delivery device such as a drive sleeve, and member 1710, e.g. a number sleeve or dial sleeve (a member with markings such as numbers indicating the currently set dose). Member 1700 is depicted as a portion integral with the user interface member. This illustrates a situation where the electronic system is integrated into the device. However, we note that the user interface member might also be connected to the member 1700, e.g. axially and rotationally locked relative to that member. This may be the case for an electronic system which is provided as an add-on or also for a system integrated into the device, e.g. for manufacturing reasons. In the position depicted in FIG. 4A, members 1700 and 1710 may be rotationally locked to one another. Axial movement of the member 1700 relative to the other member 1710 by d c may release the rotational lock such that rotational movement of the member 1710 is allowed relative to the housing (not shown which may laterally surround member 1710, in this case), relative to yet another member of the dose setting and drive mechanism and/or relative to the user interface member 1600.

In the depicted embodiment, the force which the user exerts on to the movable member 1670 is transferred to the drive mechanism member 1700 and/or clutch biasing member 1690, even before the user touches the delivery surface 1620. Consequently, there is a risk that the drive mechanism member 1700 is moved significantly by the movement of the movable member which preferably is performed solely for the purpose user proximity detection. In order to reduce the risk that the movement of the drive mechanism member 1700, e.g. in the distal direction, is too significant, the clutch biasing member 1690 and the biasing member 1680 can be adjusted to one another such that the clutch biasing member 1690 has a greater spring strength or greater spring force than the biasing member 1680. The clutch biasing member 1690 may be designed with a force greater than 1 N, e.g. in the range 1 N-3 N, when compressed, e.g. when the clutch has switched the state. The force to move the movable member into the operation position or to retain it in that position may be lower, e.g. 0.5 N.

By appropriately configuring the system, it can be ensured that the switch 1310 is triggered/the use signal is generated, before a movement of the two members 1700 and 1710 by the distance d_{c} has occurred. Thus, the use signal may be generated when the two members have already begun to move relative to one another but by less than the required distance for releasing the clutch engagement.

The system is expediently designed such that the minimum force which has to be exerted on the delivery surface in order to perform a dose delivery operation is greater than the force which has to be exerted on the movable member to trigger the use signal.

FIGS. 4A through 4C illustrate the operation of the system described in conjunction with FIG. 4A by showing different stages of the operation. In FIG. 4A the system is in a configuration, where a dose delivery operation can be initiated, e.g. when a dose has been set in the previous dose setting operation. In the case of a dial extension type device, the user interface member 1600 has been displaced proximally relative to a housing (not shown) by a distance which is proportional to the size of the set dose. In a non-dial extension type device, the user interface member may be in the same axial position relative to the housing 10 than at the stage before the dose setting operation was commenced. Thus, in FIG. 4A the system/the device is in a condition before the user intends to perform a delivery operation. When the user attempts to initiate the dose delivery operation, s/he approaches the delivery surface 1620 in order to move the user interface member 1600 for the delivery operation (at least by distance d_{c}). Before this movement is performed, the user reaches the movable member 1670, touches the contact surface 1675 and moves the movable member from the first or initial position relative to the delivery surface 1620 into the second or operation position, where the operation position is depicted in FIG. 4B. There is no movement or at least no significant movement of the user interface member relative to the housing 10 or another component 1710 of the dose setting and drive mechanism while the movable member is displaced. The dose setting and drive mechanism is still in its dose setting configuration. While moving the movable member 1670 towards or into the operation position, the use signal is generated which is symbolized by the closed sensor or switch symbol 1310. Also, the biasing member 1680 is biased and tends to move the movable member proximally or towards the initial position. The portion of the movable member which has been contacted by the user may be flush or sub-flush relative to the delivery surface such that at least the predominant part of the user force for the delivery operation is exerted onto and reacted by the delivery surface (this reduces the risk of damaging the electronics in the system). When the movable member has been moved into the operation position, the user touches the delivery surface and the movable member and the user interface member may be moved distally to initiate the delivery operation by switching the mechanism to the dose delivery configuration. This movement involves a release of a clutch engagement which is symbolized in FIG. 4C by the movement of the user interface member body and the movable member towards 10, 1710 which has occurred by the distance d_{c}. The clutch biasing member 1690 is compressed during this movement. From the situation in FIG. 4C, the dose delivery operation can be performed by transferring user force from the user interface member (body) to the piston rod (not explicitly shown) via the dose setting and drive mechanism. When the user releases the user interface member 1600 and, particularly, its delivery surface, e.g. after having completed the delivery operation for the set dose, the movable member 1670 is moved proximally back towards its initial position relative to the delivery surface by the biasing member 1680 and, again, protrudes proximally from the delivery surface 1620 as shown in FIG. 4A. Also, the clutch biasing member 1690 relaxes and re-establishes the clutch interface and the system/the device is in the situation of FIG. 4A once again. Then, the electronic system can be powered down and is ready to be woken again for a subsequent delivery operation via the signaling unit (switch 1310).

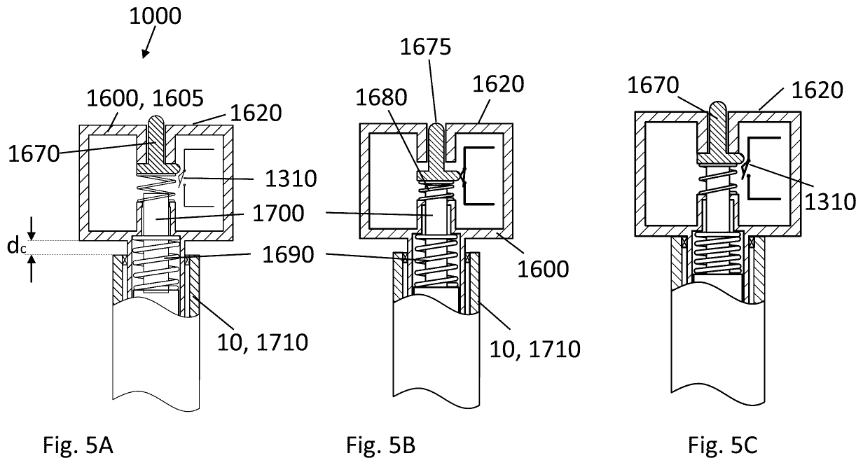

FIGS. 5A through 5C illustrate another embodiment of the electronic system. This system is very similar to the system which has been discussed above in conjunction with FIGS. 4A through 4C. Accordingly, the following discussion focuses on the differences. The representation in FIG. 5A corresponds to the representation in FIG. 4A, where the movable member 1670 is in the first or initial position relative to the user interface member 1600 and its delivery surface 1620. FIG. 5B illustrates the situation when the movable member has been moved into the second or operation position but the dose setting and drive mechanism is still in the dose setting configuration. FIG. 5C illustrates the situation when the mechanism has been switched to the dose delivery configuration.

In the embodiment depicted in FIGS. 5A to 5C, the system is configured such that the mechanism member 1700 and the movable member 1670 are decoupled from one another such that movement of the movable member 1670 from the initial position into the operation position relative to the delivery surface 1620 or the force for that movement is not transferred or transferable from the movable member to the drive mechanism member 1700. For this purpose, relative movement, e.g. axial, between the user interface member body 1605 and the mechanism member 1700 may be allowed, e.g. by an opening within the user interface member body through which the mechanism member 1700 protrudes. The biasing member 1680 may surround the member 1700 or be supported on the edge of the body 1605 delimiting the opening.

Starting from the initial position in FIG. 5A, at, first the movable member 1670 is moved relative to the delivery surface 1620 from the initial position into the operation position depicted in FIG. 5B. During this movement, the use signal is generated as has been discussed before and the system can be switched to the state of higher power consumption by the electronic control unit which, as in FIGS. 4A to 4C, is not explicitly shown in this embodiment. In the operation position, the movable member 1670 may be operatively connected to the mechanism member 1700, e.g. via abutment. Further movement of the user interface member 1600 to switch the dose setting and drive mechanism to the dose delivery configuration may involve relative movement, e.g. in the distal direction, between the user interface member body 1605 and the movable member, e.g. such that the movable member protrudes again from the delivery surface 1620. During this relative movement, the switch 1310 is moved relative to the movable member 1670 such that the switch 1310 may change its state again, e.g. from closed to open, which may reduce the power consumption of the system. If, from the situation depicted in FIG. 5C, where the movable member 1670 protrudes again from the delivery surface 1620 in the proximal direction, force is exerted in the distal direction, the dose delivery operation can be conducted. The movable member protrudes again from the delivery surface in the dose delivery configuration of the dose setting and drive mechanism. This gives the user the tactile feedback that he has successfully switched the system or the mechanism to the dose delivery configuration. Once the movable member and/or the user interface member is released by the user, the original situation between the members of the system depicted in FIG. 5A can be reestablished, e.g. on account of the relaxing biasing members as described before.

We note that instead of using the mechanism member 1700 to abut the movable member 1670 as depicted in FIG. 5B, an axial stop which is axially secured to the housing 10 or the member 1710 may be used to limit movement of the movable member during the switching to the dose delivery configuration and achieve the same effect of having the movable member protrude again from the delivery surface in the dose delivery configuration.

Hence, this embodiment provides a system, in which the movable member is moved to the operation position relative to the delivery surface to generate the signal and, when the user interface member body is moved to switch the dose setting and drive mechanism from the dose setting configuration to the dose delivery configuration, the user interface member body moves distally relative to the movable member such that it assumes its initial position relative to the delivery surface. As noted, this gives feedback to the user on the state of the system and may also reduce power consumption of the signaling unit/switch 1310.

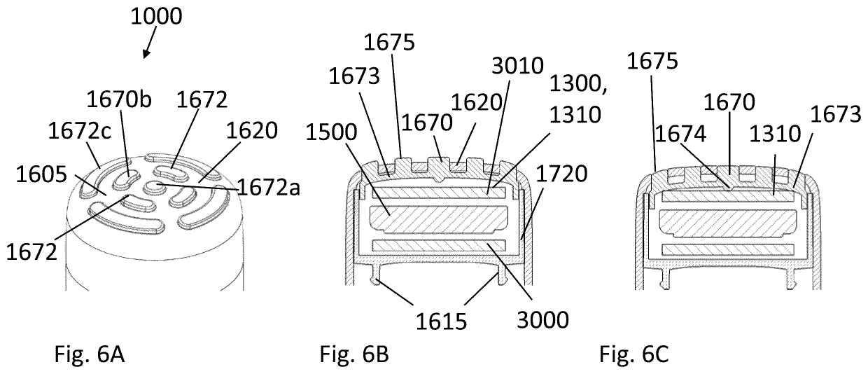

FIGS. 6A through 6C illustrate another embodiment of the electronic system. This system is very similar to the systems which have been discussed above in conjunction with FIGS. 4A to 5C. Accordingly, the following discussion focuses on the differences.

The user interface member 1600 is shown in a perspective view onto the delivery surface 1620 in FIG. 6A. FIG. 6A shows the movable member 1670 in its initial position proud of the delivery surface 1620. In this embodiment, the movable member 1670 has a plurality of portions 1672, which protrude from the delivery surface 1620 at different locations on the surface. Each portion protrudes through a dedicated opening in the user interface member body 1605 which has a circumferentially closed edge or border. Exemplary sections are denoted with 1670a through 1670c in FIG. 6A. All of the sections do belong to the same movable member 1670. Specifically, they are connected to a common main body 1673 provided in the interior of the user interface member body 1605. Accordingly, if the user touches one of the sections and moves this section relative to the delivery surface, the entire movable member will be moved towards the delivery surface. This movement can, again, be used to trigger a use signal generation in a signaling unit, e.g. a switch 1310. The movable member 1670 is expediently rigid. Thus, the movable member is not elastically deformed when being moved from the initial position to the operation position. The delivery surface or the user interface member body 1605 forming that surface are expediently rigid as well.

The portions of the movable member are disposed at various locations on the delivery surface 1620. Portions which are further from the center of the delivery surface 1620 may have a greater extension, especially in the circumferential or angular direction, than portions which are closer to the center. In the depicted arrangement, the angular or circumferential extension of portion 1670c is greater than the one of portion 1670b and/or of the portion 1670a. The radial extension of the respective portions may be constant along their angular extension and/or equal between sections located at different radial positions but at the same angular location. Portion 1672a may be arranged in the center of the delivery surface 1620. Several angularly separated rows or rays of portions 1672b and 1672c emerge from the central portion 1672a and are oriented radially. Other configurations of protruding portions 1672 are possible to ensure that the movable member is reliably moved before the user touches the surface, of course.

The electronic system 1000 or the user interface member 1600, again, may be an add-on to a device unit or may be integrated into the device. In the present embodiment, the user interface member 1600 or the electronic system 1000 is an add-on for a drug delivery device unit and comprises connection features 1615 which are designed to interact with a member of a drug delivery device unit, e.g. a dose knob or dose button, so as to rigidly, preferably axially and rotationally, lock the user interface member to the member of the device. Connection feature 1615 may be designed for a snap-fit connection for example. The connection may be releasable such that the electronic system can be used with more than one drug delivery device or permanent when the system is to be disposed of after one use cycle together with the device. The connection features 1615 are shown in FIG. 6B which shows a schematic sectional view of the electronic system in the same situation as is depicted in FIG. 6A. The connection features 1615 are expediently disposed in the interior of the user interface member body 1605. The user interface member body 1605, when the system is connected to the member of the dose setting and drive mechanism, is preferably connected to the member to transfer force or torque to the member.

The user interface member 1600 or the system further comprises a user interface member part 1720. The user interface member part 1720 is rigidly, expediently rotationally and axially, connected or fixed to the user interface member body, e.g. by snap-fit or welding. The user interface member part may be cup shaped. The user interface member part 1720 serves as a carrier for one or more electronic or electrical components in the system. The respective component may be arranged in the space delimited by the outer wall of the part 1720. As non-limiting and non-exhaustive examples, the conductor carrier 3000 with the electronic control unit arranged thereon (not shown) and the power supply 1500 are shown in FIG. 6B. The respective component may be arranged on and preferably fixed to the user interface member part 1720, e.g. on a proximal surface thereof. Electrical interconnections are not shown but can be provided within the user interface member body as desired. The connection features 1615 are provided on the user interface member part 1720 and may protrude in the distal direction. The connection features may be arranged within the hollow defined by the user interface member body.

As is apparent from the sectional view in FIG. 6B, a signaling unit 1300, particularly a switching mechanism 1310, is associated with the movable member 1670. The switching mechanism may be a switch such as a force switch. The signaling unit may be arranged on a conductor carrier 3010, e.g. a circuit board, different from the conductor carrier 3000. The power supply may be arranged between the conductor carriers 3000 and 3010. The conductor carrier 3010 is expediently arranged closer to the exterior operation surface than the conductor carrier 3000. Both conductor carriers may be rigid. The respective conductor carrier 3000, 3010 may be axially and rotationally secured relative to the user interface member body 1605, e.g. to the part 1720. The signaling unit 1300 is expediently conductively connected to the electronic control unit 1100 which may be provided on the conductor carrier 3000.

If the user moves the movable member 1670 towards the delivery surface 1620 which corresponds to a movement in the distal direction in the depicted embodiment, the switch 1310 is triggered. For triggering the switch 1310 a mechanical contact is established between the movable member 1670 e.g. a distally oriented protrusion thereof, and the switch 1310.

A shape of the enveloping surface defined by the portions 1672 of the movable member 1670 is matched to the shape of the enveloping surface of the delivery surface 1620 in this embodiment. The radial extension of the respective portion 1672 is expediently chosen such that, if the movable member 1670 has been moved relative to the delivery surface 1620, the users finger mainly contacts the delivery surface and/or the primary load of the user acting on the user interface member is reacted by and transferred to the delivery surface 1620 and, preferably, not to the movable member 1670. Alternatively or additionally, there is no distal end stop to define the operation position in the system as described above already.

In the situation when the movable member 1670 has been moved distally into the operation position (see FIG. 6C), the switch 1310 has been triggered. The movable member may have a switching feature 1674, which protrudes distally from the interior surface of the main body 1673, for interacting with the switch 1310. The switch, when triggered, provides the use signal which causes the electronic control unit 1100 (not shown in this representation) to switch the system to the second state of higher power consumption as outlined further above. In the operation position the contact surface 1675 between the movable member and the user (i.e. the proximally facing surface of the portion(s) of the movable member protruding from or being proud of the delivery surface 1620) is recessed relative to the delivery surface 1620, e.g. by at least 0.1 mm or at least 0.2 mm and/or by at most 0.8 mm or at most 0.7 mm, e.g. by 0.5 mm. This assists in exerting the primary load for the delivery operation onto the delivery surface and not the movable member 1670 expediently along with a missing end stop. This can be seen in FIG. 6C. Again, the movable member may be biased towards its initial position depicted in FIG. 6A, e.g. by a biasing member which is not explicitly shown in FIG. 6, but may be present.

The respective portion 1672 of the movable member 1670 which protrudes from the delivery surface may be delimited from all the other portions of the movable member protruding from the delivery surface. In other words, the protruding portions are expediently separated on the exterior of the user interface member (body) and preferably connected in the interior of the user interface member (body) to the main body 1673.

The main body may have a sealed interface with interior wall(s) of the user interface member, e.g. walls of the interface member body 1605 and/or the interface member part 1720. For this purpose, a sealing member (not shown), e.g. an o-ring, may be provided along the entire outer circumference of the main body 1673. In this way, the compartment of the user interface member interior which retains electrical or electronic components or units may be sealed relative to the exterior, e.g. against the ingress of moisture or dirt, despite the provision of the movable member, via the opening(s) in the delivery surface. Although it is generally envisaged that features of different embodiments can be combined with each other unless related to mutually exclusive solutions, we note explicitly that such a sealing member may also be provided on the movable member, e.g. on its main body, in the other embodiments discussed herein.

In this embodiment the switch 1310 is mounted beneath the movable member and preferably fixed axially and/or rotationally relative to the user interface member body 1605. The switch 1310 preferably has a very light operating force to ensure that the switch operates prior to the axial disengagement of the clutch interface for switching the dose setting and drive mechanism from the dose setting configuration to the dose delivery configuration as discussed already. A low-force micro-switch may be used as switch 1310. The force required to act on the movable member to trigger the switch may be smaller than the force which has to be overcome to switch the clutch interface, e.g. from established (clutch features are engaged) to released (clutch features are disengaged) or vice versa. As noted already, the force to switch the clutch interface may be 1 to 3 N. The force to trigger the switch or displace the movable member may be smaller, e.g. 0.5 N.

In its initial or first position, the movable member 1670 presents a plurality of contact surfaces 1675 proud of the top surface or delivery surface 1620 of the user interface member body 1605. In its operation or second position, these contact surfaces 1675 become sub-flush relative to the deliver surface or the body 1605. Each contact surface 1675 is preferably sufficiently small such that once the movable member has travelled axially and operated the signaling unit (by triggering switch 1310), the user's finger, e.g. the thumb, will then bear down only or predominantly on the delivery surface and not on the contact surfaces. This ensures or assists in that the primary load path from the user's finger to the user interface member, and subsequently to the dose setting and drive mechanism does not pass through the electronic components, e.g. through the switch, and instead passes through the rigid body or a component retained therein designed for transferring the delivery force required to operate the device for conducting the delivery operation.

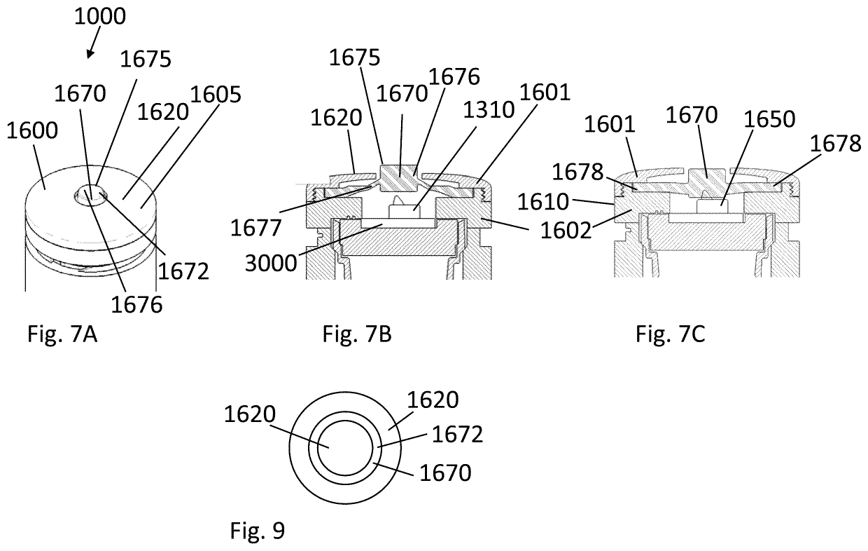

FIGS. 7A through 7C illustrate another embodiment of the electronic system. This embodiment is very similar to the systems which have been discussed above in conjunction with FIGS. 4A to 6C. Accordingly, the following discussion focuses on the differences.

FIG. 7A, shows a perspective view onto the delivery surface 1620 of the user interface member 1600. Again, as in the previous embodiments, a movable member 1670 is provided.

Specifically, at least one portion 1672 thereof (in the depicted embodiment just one portion but a plurality of portions are also possible) protrudes through an opening in the delivery surface 1620 and provides a contact surface 1675 or pad for being contacted by the user. The contact surface 1675 in the initial position (FIGS. 7A and 7B) is proud of and/or is proximally offset from the delivery surface 1620. The previous embodiments relied on a rigid movable member 1670. In contrast thereto, the present embodiment utilizes a deformable, particularly elastically deformable, movable member 1670. In this embodiment, the movable member 1670 may be elastomeric. The movable member can be deformed or flexed to contact the switch 1310 before or when moving flush or sub-flush relative to the delivery surface 1620 to assume the operation position (see FIG. 7C). Thus, when the user contacts the delivery surface, the movable member is in the operation position and the use signal has been provided by the signaling unit 1300 comprising the switch 1310.

The movable member 1670 expediently is an elastically deformable member. Thus, the member can be elastically deformed and, when it is deformed, it tends to restore its undeformed shape due to an elastic restoring force. Accordingly, if an elastically deformable movable member 1670 is used, a separate biasing member (see biasing member 1680 discussed further above) can be dispensed with and the elastic restoring force can be used for establishing the initial position of the movable member. The member 1670 is unitary, for example. In other words, all portions may be of the same material.

As is apparent from the cross-sectional view in FIG. 7B, the movable member 1670 has a protruding contact portion 1676 which may form the contact surface 1675. Contact portion 1676, preferably, is more rigid and/or has a greater thickness along the axial direction than one or more other portions of the member 1670, e.g. one or more interior portions within the user interface member body in the initial position. The axial direction may be the direction which separates the initial and operation positions of the movable member, e.g. the distal direction. Connected to the contact portion 1676, e.g. adjacent such as radially adjacent, are one or more elastically deformable portions 1677 of the movable member. The deformable portion(s) 1677 may be always arranged in the interior of the user interface member body 1605, i.e. in the operation position of the movable member or its contact surface 1675 and in the initial position. When the moveable member is moved towards the operation position the deformable portion(s) may be flexed and deformed to a greater extent than the contact portion 1676. The contact portion 1676, with an interior or distal surface thereof, is arranged to cooperate with the switch 1310 which is arranged on the conductor carrier 3000 which is expediently axially fixed relative to the delivery surface 1620. Other arrangements for the signaling unit are possible, e.g. similar as in FIGS. 6A to 6C. Thus, the contact portion 1676 can trigger the switch 1310 when the member 1670 is displaced towards the operation position which is shown in FIG. 7C in order to provide the signal. The force required to elastically deform the movable member 1670 to move the member 1670 into the operation position is expediently smaller than the force required to switch the mechanism from the dose setting configuration to the dose delivery configuration.

Using an elastomeric member, e.g. of rubber or similar material, for the movable member provides the possibility to seal electronic components tightly in the system using the movable member to establish the sealing. For this purpose, the movable member 1670 may sealingly engage one or more interior surfaces of the user interface member (body). The sealing engagement may be strengthened by mechanical forces which keep the contact regions between the interior surface and the movable member under the influence of compressive force to maintain a tight seal. The seal may protect the interior components such as electronic components from water and dirt ingress. In the depicted embodiment the movable member comprises a sealing portion 1678. The sealing portion 1678 is an edge portion of the movable member which delimits the movable member 1670, expediently laterally or radially. The sealing portion 1678, preferably, is a radial end portion of the movable member. In the depicted embodiment, the sealing portion 1678 extends circumferentially around the movable member. The deformable portion 1677 may extend circumferentially around the contact portion 1676. The sealing portion 1678 is connected to the contact portion 1676 via the deformable portion 1677. The sealing portion 1678 is preferably clamped between two parts 1601 and 1602 of the user interface member (body). The parts 1601 and 1602 may maintain the sealing portion 1678 in a deformed or clamped condition when the movable member has been assembled to the user interface member body. The parts 1601 and 1602 can be connected to each other, e.g. via threads as hinted in FIGS. 7B and 7C. Both parts may belong to the user interface member body. In this case, both parts may form at least a section of the exterior surface of the user interface member. In the present embodiment, part 1601 forms the delivery surface 1620 (and potentially a part of the setting surface 1610) and part 1602 forms the setting surface 1610 (at least a part thereof). Alternatively, one part may be an interior part of the user interface member or both parts may be interior parts. Thus, the compartment of the interior of the user interface member 1600 which is arranged on that side of the movable member remote from the delivery surface 1620 is sealed with respect to the delivery surface. Again, the opening in the delivery surface 1620 through which the movable member protrudes is expediently configured to be small enough such that at least the majority of the force which the user provides during a dose delivery operation is reacted by the delivery surface and not by the movable member. This protects the components in the interior of the user interface member from higher loads.

In this embodiment the contact surface 1675, preferably a small surface, is proud of the delivery surface in the initial position. The contact surface is designed to be displaced, preferably again at a low force, and operate the switch 1310, e.g. a micro-switch. When fully deflected, the movable member is designed to be sub-flush to the delivery surface 1620 of the user interface member body. The flexible surface area is expediently sufficiently small that the user's finger, e.g. the user's thumb, will bear against the surrounding rigid delivery surface once the movable member has been deflected to be sub-flush with respect to that surface sub-flush. The contact area is preferably sufficiently small such that once deflected, the user will then bear down only or at least predominantly on the delivery surface of the user interface member body and not on the contact surface of the movable member. This ensures the primary load path generated by the user during delivery does not pass through electronics, e.g. the switch, and instead passes through the rigid body into the dose setting and drive mechanism of the device. The elastomeric movable member further gives the opportunity to form a sealed assembly, protecting electronic components and their periphery, e.g. a printed circuit board or conductors, such as from water and/or dirt ingress without requiring a separate sealing member, such as an o-ring.

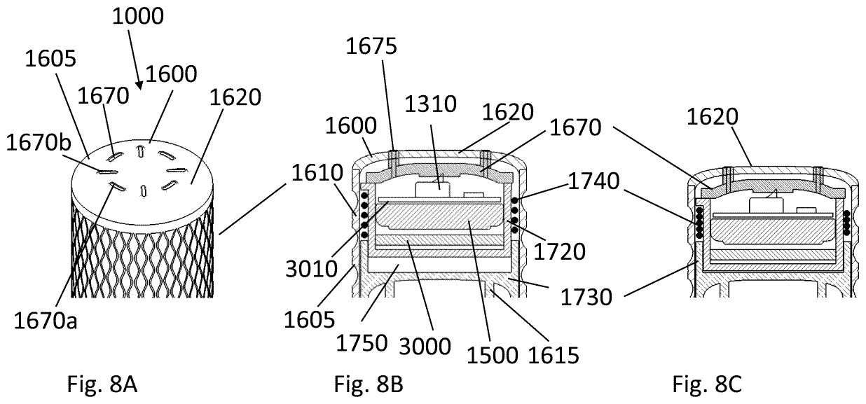

FIGS. 8A through 8C illustrate another embodiment of the electronic system 1000. This system is very similar to systems which have been discussed above in conjunction with FIGS. 4A through 7C. Accordingly, the following discussion focuses on the differences.

FIG. 8A shows a perspective view onto the delivery surface 1620. The movable member 1670 comprises a plurality of protruding portions (eight portions in the depicted embodiment) in the initial state as depicted in FIG. 8A. Portions 1670a and 1670b are highlighted, because they are oriented in different directions. The portion 1670a is oriented in the angular direction. The portion 1670b is oriented in the radial direction. Radially and angularly oriented portions of the movable member 1670 may alternate in the circumferential or angular direction. It should be appreciated that the arrangement of the sections of the movable member 1670 which protrude from the delivery surface 1620 can also be different as could their number.

As opposed to the previously discussed embodiments, in this embodiment, a shuttle member 1730 is provided. The shuttle member 1730 is preferably a part of the electronic system and movably connected to the user interface member body 1605 and/or the exterior operation surface or delivery surface 1620. The shuttle member 1730 may be permanently retained in the user interface member body. A distal end stop limiting distal movement of the shuttle member relative to the user interface member body may be provided for this purpose. The shuttle member 1730 is expediently provided to be arranged in the force transfer path between the exterior operation surface (delivery surface 1620) and a member of the dose setting and drive mechanism, e.g. the mechanism member which has been discussed further above such as the drive sleeve or the injection button. If the electronic system is an add-on module for a drug delivery device unit, the shuttle member 1730 may be that member of the electronic system which is configured to have the connection feature 1615 for connecting the electronic system to the drug delivery device unit. Alternatively, the shuttle member may be the mechanism member, especially if the electronic system is integrated into the drug delivery device. The shuttle member 1730 when operatively connected to the dose setting and drive mechanism is axially and preferably rotationally locked relative to the member of the dose setting and drive mechanism to which it is connected. Particularly, the axial force transferred from the exterior operation surface 1620 to the shuttle member 1730 may result in the force being transferred to the dose setting and drive mechanism, particularly for the dose delivery operation, via the shuttle member. The shuttle member is preferably rotationally locked to the user interface member body 1650. In this way, dial torque may be transferred to the mechanism member from the setting surface 1610. The shuttle member may have a continuous distal surface. An interface between the shuttle member and the 1730 and an inner wall of the user interface member body 1605 is preferably sealed, e.g. via an o-ring.

A shuttle biasing member 1740 is operatively arranged between the interface of electronic system with the dose setting and drive mechanism and the exterior operation surface (delivery surface 1620) or the movable member 1670. The shuttle biasing member 1740, e.g. a compression spring, such as a helical spring, may be arranged such that it has to be biased, e.g. compressed, before force can be transferred from delivery surface 1620 to the dose setting and drive mechanism for the delivery operation. This facilitates that the movable member 1670 has always been displaced into the operation position or the signal has been generated before the dose setting and drive mechanism can be operated, e.g. before being switched from the dose setting configuration to the dose delivery configuration. For example, the biasing member 1740 may be operatively coupled between the shuttle member 1730 and the exterior operation surface (delivery surface 1620). The biasing force or pre-load of the shuttle biasing member 1740, e.g. when fully compressed and/or when the movable member is in the operation position, is expediently smaller than the force provided by the clutch biasing member in the dose setting and drive mechanism, e.g. when the clutch interface is released (see the description further above). In the depicted embodiment, as is apparent from FIG. 8B, the biasing member 1740 is operatively arranged between the user interface member part or carrier 1720 and the shuttle member 1730. The carrier 1720 may be provided as a carrier for one or more electronic components or units of the system. In the depicted embodiment, the carrier may serve as a carrier for the conductor carrier 3000 (with the electronic control unit 1100), the power supply 1500, and/or the conductor carrier 3010 with the signaling unit 1300, which is represented by the switch 1310. The recited components or units may be axially secured to the carrier 1720. The carrier 1720 may be axially and rotationally secured to the user interface member body 1605. The carrier 1720 corresponds to the user interface member part of the embodiment described in conjunction with FIG. 7A to 7C.

FIG. 8B shows the situation, when the movable member 1670 has been moved into the operation position. The member may be biased towards its initial position as discussed previously, where the biasing member is not shown in this embodiment. The operation position may be defined by an abutment or axial end stop of the movable member 1670 relative to the user interface member body or the exterior operation surface. In the present embodiment this end stop is provided by the carrier 1720. However, we note that the end stop could also be provided by another component or system. The contact surface 1675 is still proud of the delivery surface in the operation position but protrudes less from that surface than in the initial position. Alternatively, the contact surface 1675 may be flush or sub-flush relative to the delivery surface 1620. In this position, which is before the dose setting and drive mechanism has been switched from the dose setting configuration into the dose delivery configuration, the biasing member 1740 still needs to be biased before the force exerted on the exterior operation surface can be transferred to the shuttle member via an axial abutment, e.g. between the carrier 1720 and the shuttle member 1730 in region 1750 (see the free space between carrier and shuttle member in FIG. 8B and the abutment in FIG. 8C). Thus, when the movable member is in the operation position as in FIG. 8B, the biasing member 1740 still has to be biased before the dose setting and drive mechanism can switch its configuration to the dose delivery configuration in order to drive the dose delivery operation. The biasing member 1740 may provide a known preload which has to be overcome by the user before the mechanism can be switched to the dose delivery configuration. The biasing member may be biased in the initial position of the movable member already. The biasing member 1740 may bias the delivery surface 1620 away from the shuttle member in the initial position of the movable member. The biasing member may be dimensioned or configured such that the use signal is generated/the switch 1310 is triggered before the mechanism is switched to the dose delivery configuration. The user, however, has to act against the biasing member 1740 before a driving engagement or dose delivery interface can be established with the shuttle member and/or the mechanism member.

The carrier 1720 may serve as or comprise a light guide for guiding light originating from an optoelectronic light source of the motion sensing unit towards a sensing surface or encoder surface on the mechanism member which movement should be monitored by way of the motion sensing unit. The motion sensing unit, expediently, is arranged on the conductor carrier 3000. We note that the arrangement of the power supply and the conductor carriers 3000 and 3010 is just an example of one possible implementation of arranging the components in the user interface member. This implementation may be optimized with respect to space requirements such that the components may fit into a user interface member 1600, e.g. one with the dimensions specified further above.

When from the situation in FIG. 8B, the delivery surface is displaced distally relative to the shuttle member, the biasing member 1620 is further biased, e.g. compressed, and the situation depicted in FIG. 8C emerges where the force transfer engagement has been established by the carrier 1720 abutting the shuttle member 1730 to transfer the delivery force from the delivery surface to the dose setting and drive mechanism.

The increase in the user force (required to bias the biasing member 1740) is tolerable, as this guarantees that, in all tolerance conditions (considering tolerances in the dose setting and drive mechanism and also in the electronic system), the signal is generated before the dose delivery operation is commenced, e.g. before one mechanism member is moved relative to the other mechanism member to switch the clutch interface or at least before the clutch interface is switched. Once the bias spring force of the biasing member is overcome, any additional force provided by the user acts to drive the dose delivery operation.

As in previous embodiments, the switch 1310 is mounted beneath the movable member 1670 which is designed to travel relative to one or more of the electronic components of the system, e.g. relative to the switch when the user applies a distal load to the delivery surface 1620. The switch, e.g. a micro switch, expediently has a very light operating force to ensure that the switch operates prior to the disengagement or releasing of the clutch in the device. To ensure that the switch is reliably able to operate at a lower force than the force to axially displace the injection button or another component of the dose setting and drive mechanism of the device, the shuttle member 1730 and the biasing member are added as opposed to the previous embodiments.

The shuttle member 1730, when connected to the dose setting and drive mechanism may be biased axially (in this embodiment with the biasing member 1740) in the distal direction relative to delivery surface 1620. The biasing member 1740 may be configured to provide a pre-load in all tolerance conditions to bias the user interface member body 1605, carrier 1720 and/or the electronic components proximally with a known force. The system is expediently designed such that in all tolerance conditions, the connection of the electronic system to the dose setting and drive mechanism, e.g. to a dose button thereof, will cause some compression of the shuttle member against the biasing member 1740, thereby generating a (known) preload.

The user interface member 1600 cannot move axially and operate the drive mechanism until the pre-load is exceeded. Movement of the user interface member relative to the shuttle member without operation the dose setting and drive mechanism by moving a mechanism member is allowed. The movable member 1670 can be reliably designed to engage the switch 1310 at a force lower than the preload, ensuring that the dose delivery operation is not initiated or commenced until the switch has been operated. This concept can be realized with either an elastomeric or a rigid movable member.

As an alternative to the shuttle member being movable relative to the user interface member body, where the signaling unit is fixed relative to the user interface member body, the exterior operation surface may be movable relative to the signaling unit when the user interface member body is moved for switching the mechanism into the dose delivery configuration. In this case, the shuttle member may be a member fixed to the user interface member body. The biasing member 1740 may bias the carrier 1720 towards the operation surface 1620. For generating the signal via the switch 1310, the movable member 1670 needs to be in the operation position. Additionally, a movement of the exterior operation surface towards the signaling unit or the switch 1310 may be required.

The embodiments which have been described previously often used portions of the movable member 1670 which protruded from the exterior operation surface in the initial position, where the portions covered a continuous area of the surface of the user interface member when seen in top view onto the delivery surface 1620. That is to say, none of the portions of the movable member did encircle a region of the delivery surface 1620 when seen in top view onto that surface.

FIG. 9 illustrates an embodiment, where the protruding portion 1672 of the movable member 1670 on the delivery surface 1620 has a ring-like configuration, using a top view onto the delivery surface. Thus, this portion entirely encircles a region of the delivery surface. This emphasizes that different configurations of the protruding portion(s) are possible. The remaining operation principles which have been described previously remain for this embodiment.

We note that the embodiments which have been disclosed above are not all of the possible embodiments. Embodiments depicted above enable generation of the signal for triggering the switching of the system to the state of higher power consumption when the movable member has reached the operation position or at least before the dose setting and drive mechanism is switched to the dose delivery configuration, potentially after the exterior operation surface and/or the movable member has been displaced in the distal direction relative to the signaling unit and/or the mechanism member.

The skilled person will appreciate that various combinations of features from different embodiments are within the disclosure, especially if the combination is not explicitly ruled out by contradictions between the embodiments.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The scope of protection is not limited to the examples given herein above. Any invention disclosed herein is embodied in each novel characteristic and each combination of characteristics, which particularly includes every combination of any features which are stated in the claims, even if this feature or this combination of features is not explicitly stated in the claims or in the examples.

### REFERENCE NUMERALS

1 injection device, drug delivery device or device unit
10 housing
12 dosage knob
11 injection button
13 window
14 container
15 needle
16 inner needle cap
17 outer needle cap
18 cap
70 dial or number sleeve
71a-c formation
1000 electronic system
1100 electronic control unit
1200 motion sensing unit
1300 signaling unit
1310 switch
1400 communication unit
1500 electrical power supply
1600 user interface member
1605 user interface member body
1610 setting surface
1615 connection feature
1620 delivery surface
1670 movable member
1671 feature
1672 portion
1672a portion
1672b portion
1672c portion
1673 main body
1674 switching feature
1675 contact surface
1676 contact portion
1677 deformable portion
1678 sealing portion
1680 biasing member
1690 clutch biasing member
1700 member
1710 member
1720 user interface member part
1730 shuttle member
1740 biasing member
1750 region
3000 conductor carrier
3010 conductor carrier
d_{c} distance

1-19. (canceled)

20. An electronic system for a drug delivery device, the electronic system comprising:
at least one user interface member configured to be manipulated by a user for performing a dose operation,
an electronic control unit configured to control operation of the electronic system, the electronic system having a first state and a second state, wherein the electronic system has an increased electrical power consumption in the second state as compared to the first state,
wherein the user interface member comprises an exterior operation surface which is arranged to interact with the user for the dose operation,
wherein the user interface member comprises a user proximity detection unit configured to generate an electrical signal in response to detecting that the user is close to the exterior operation surface or touches the exterior operation surface,
wherein the user proximity detection unit comprises a movable member arranged to be moved by the user away from an initial position relative to the exterior operation surface towards an operation position before the user reaches the exterior operation surface,
wherein the user proximity detection unit further comprises an electrical signaling unit, wherein the user proximity detection unit is configured to provide the electrical signal when the movable member has been moved away from the initial position, and
wherein the electronic system is configured such that the electronic control unit switches the electronic system from the first state into the second state in response to the electrical signal.

21. The electronic system of claim 20,
wherein the user interface member comprises a user interface member body, wherein the user interface member body defines the exterior operation surface, and wherein the exterior operation surface delimits at least one opening, wherein a portion of the movable member protrudes through the opening.

22. The electronic system of claim 21,
wherein the exterior operation surface delimits a plurality of separate openings, wherein a portion of the movable member protrudes through each opening, wherein the portions originate from a common main body of the movable member which is provided in an interior of the user interface member body.

23. The electronic system of claim 21,
wherein, when the movable member is in the operation position, a force exerted on the movable member is resolved by the user interface member body via a force transfer path which bypasses the electrical signaling unit and/or the electronic control unit.

24. The electronic system of claim 20,
wherein, in the initial position of the movable member, a user contact region of the movable member is elevated relative to the exterior operation surface, and wherein, in the operation position of the movable member, the user contact region is sub-flush relative to the exterior operation surface.

25. The electronic system of claim 20,
wherein the electrical signal is generated in response to movement of the movable member relative to the electrical signaling unit, and/or
wherein the electrical signaling unit comprises an electrical switch, wherein the electrical switch is arranged to be triggered during the movement from the initial position to the operation position.

26. The electronic system of claim 20,
wherein the movable member, when it is in the operation position, is biased towards the initial position by a member biasing mechanism.

27. The electronic system of claim 20,
wherein the exterior operation surface is a delivery surface configured to interact with the user for performing a dose delivery operation as the dose operation.

28. The electronic system of claim 20,
wherein the user interface member is connected to or configured to be connected to a mechanism member of a dose setting and/or a drive mechanism of the drug delivery device such that force can be transferred from the exterior operation surface to the mechanism member.

29. The electronic system of claim 28,
wherein the dose operation is a dose delivery operation, wherein the mechanism member is a second member and wherein the dose setting and the drive mechanism comprises a first member, wherein the first member and the second member are configured to be movable relative to each other for switching the dose setting and the drive mechanism from a dose setting configuration to a dose delivery configuration, and wherein the electronic system is configured such that the electrical signal is generated before the dose setting and the drive mechanism is switched from the dose setting configuration to the dose delivery configuration.

30. The electronic system of claim 20,
wherein the electronic system comprises a shuttle member that is movable relative to the exterior operation surface, and wherein the electronic system comprises a shuttle member biasing system configured to bias the shuttle member away from the exterior operation surface and/or the movable member.

31. The electronic system of claim 30,
wherein the electronic system is configured such that the user interface member is operatively connectable to a mechanism member via a dose operation interface to drive movement of the mechanism member during the dose operation and wherein the electronic system is configured such that, in order to establish the dose operation interface, the exterior operation surface has to be displaced from a first position relative to the mechanism member to a second position relative to the mechanism member, and wherein the shuttle member biasing system is configured to be biased during the movement from the first position to the second position.

32. The electronic system of claim 20,
wherein the electronic system is configured as an add-on module for a drug delivery device unit.

33. The electronic system of claim 20,
wherein, as seen in plan view or top view onto the exterior operation surface, an area covered by the exterior operation surface is greater than an area covered by the movable member, and
wherein a ratio between the area covered by the movable member and the area of the exterior operation surface is less than or equal to 0.4.

34. The electronic system of claim 20,
wherein, when the movable member is in the operation position, the movable member is configured to be moved further away from the initial position.

35. The electronic system of claim 20,
wherein the electrical signaling unit is secured relative to the exterior operation surface against movement away from and/or towards the exterior operation surface.

36. The electronic system of claim 20,
wherein the movable member is configured and arranged relative to the exterior operation surface such that the movable member has to be moved before the user touches the exterior operation surface.

37. A drug delivery device comprising an electronic system, the electronic system comprising:
at least one user interface member configured to be manipulated by a user for performing a dose operation,
an electronic control unit, the electronic control unit being configured to control operation of the electronic system, the electronic system having a first state and a second state, wherein the electronic system has an increased electrical power consumption in the second state as compared to the first state,
wherein the user interface member comprises an exterior operation surface which is arranged to interact with a user for the dose operation,
wherein the user interface member comprises a user proximity detection unit, wherein the user proximity detection unit is configured to generate an electrical signal in response to detecting that the user is close to the exterior operation surface or touches the exterior operation surface,
wherein the user proximity detection unit comprises a movable member, wherein the movable member is arranged to be moved by the user away from an initial position relative to the exterior operation surface towards an operation position before the user reaches the exterior operation surface,
wherein the user proximity detection unit further comprises an electrical signaling unit, wherein the user proximity detection unit is configured to provide the electrical signal when the movable member has been moved away from the initial position, and
wherein the electronic system is configured such that the electronic control unit switches the electronic system from the first state into the second state in response to the electrical signal.

38. The drug delivery device of claim 37, further comprising a reservoir with a drug.

39. A method comprising:
dispensing a drug with a drug delivery device, wherein the drug delivery device comprising a reservoir with the drug and an electronic system, wherein the electronic system comprises:
at least one user interface member configured to be manipulated by a user for performing a dose operation,
an electronic control unit, the electronic control unit being configured to control operation of the electronic system, the electronic system having a first state and a second state, wherein the electronic system has an increased electrical power consumption in the second state as compared to the first state,
wherein the user interface member comprises an exterior operation surface which is arranged to interact with a user for the dose operation,
wherein the user interface member comprises a user proximity detection unit, wherein the user proximity detection unit is configured to generate an electrical signal in response to detecting that the user is close to the exterior operation surface or touches the exterior operation surface,
wherein the user proximity detection unit comprises a movable member, wherein the movable member is arranged to be moved by the user away from an initial position relative to the exterior operation surface towards an operation position before the user reaches the exterior operation surface,
wherein the user proximity detection unit further comprises an electrical signaling unit, wherein the user proximity detection unit is configured to provide the electrical signal when the movable member has been moved away from the initial position, and
wherein the electronic system is configured such that the electronic control unit switches the electronic system from the first state into the second state in response to the electrical signal.
